(19) United States
(12) Patent Application Publication
(10) Pub. No.: US 2026/0283911 A1
(43) Pub. Date: Sep. 24, 2026

(54) DENTAL COMPOSITIONS AND RELATED METHODS AND DEVICES

(71) Applicant: ZEST IP HOLDINGS, LLC, Carlsbad, CA (US)

(72) Inventors: Yuping Li, Irvine, CA (US); Nicholas Aralis, San Juan Capistrano, CA (US); Wen Gu, Riverside, CA (US)

(21) Appl. No.: 19/574,152

(22) Filed: Mar. 20, 2026

Related U.S. Application Data
(60) Provisional application No. 63/775,943, filed on Mar. 21, 2025.

(51) Int. Cl. A61K 6/887; A61K 6/70; A61K 6/889

(52) U.S. Cl. CPC A61K 6/887; A61K 6/70; A61K 6/889

(57) ABSTRACT

An aspect of the disclosure is related to a dental composition comprising a liquid carrier, a thickening agent to increase viscosity of the dental composition, and an active agent. The composition can have a viscosity sufficient to increase residence time of the composition on a tooth surface and reduce uncontrolled spreading relative to an otherwise identical composition lacking a thickening agent. The dental composition can be in the form of a homogeneous gel and is capable of remaining homogeneous for at least about 8 weeks. The dental composition exhibits relatively more storage stability compared to the dental composition without the thickening agent.

### CROSS-REFERENCE TO RELATED APPLICATION

This application claims priority to U.S. Provisional Application Ser. No. 63/775,943, filed on Mar. 21, 2025. This application and all other extrinsic materials discussed herein, including publications, patent applications, and patents, are incorporated by reference in their entirety. Where a definition or use of a term in an incorporated reference is inconsistent or contrary to the definition of that term provided herein, the definition of that term provided herein applies and the definition of the term in the reference does not apply.

### BACKGROUND

Dental compositions can include materials used in restorative dentistry to repair or replace tooth structure. Dental compositions can include various types of composites, amalgams, ceramics, and cements, each with unique properties suited for specific dental applications. For example, aqueous dental compositions can include dental materials that utilize water as a solvent or dispersing medium, enhancing their handling and application properties. These compositions often include a variety of components such as water-soluble polymers, fillers, and active ingredients designed for dental treatments.

### SUMMARY

This Summary is not intended to identify key aspects or essential aspects of the claimed subject matter.

All features of exemplary embodiments which are described in this disclosure and are not mutually exclusive can be combined with one another. Elements of one embodiment can be utilized in the other embodiments without further mention. Other aspects and features of the present disclosure will become apparent to those ordinarily skilled in the art upon review of the following description of specific embodiments in conjunction with the accompanying Figures.

The present disclosure relates to a dental composition including: a liquid carrier including water and at least one organic liquid selected from the group consisting of an alcohol, a polyol, an olefinic monomer, glycerin, propylene glycol, polyethylene glycol, ethanol, acetone; a thickening agent including a polyvinylpyrrolidone having vinylpyrrolidone side groups; and an active agent; wherein the dental composition is in the form of a homogeneous gel and is capable of remaining homogeneous for an extended period of time.

The present disclosure relates to a dental composition including: a liquid carrier; a thickening agent to increase viscosity of the dental composition; and an active agent.

The present disclosure relates to a dental composition, wherein the thickening agent includes a polyvinylpyrrolidone. The present disclosure relates to a dental composition, the polyvinylpyrrolidone has a vinylpyrrolidone side group. The present disclosure relates to a dental composition, the polyvinylpyrrolidone has a plurality of vinylpyrrolidone side groups.

The present disclosure relates to a dental composition, wherein the liquid carrier includes at least one organic liquid selected from the group consisting of water, an alcohol, a polyol, an olefinic monomer, glycerin, propylene glycol, polyethylene glycol, ethanol, acetone.

The present disclosure relates to a dental composition, wherein the dental composition is in the form of a homogeneous gel. The present disclosure relates to a dental composition, wherein the dental composition is capable of remaining homogeneous for a period of time.

The present disclosure relates to a dental composition, wherein the polyvinylpyrrolidone includes a homopolymer or a copolymer of one or more monomers having one or more vinylpyrrolidone groups.

The present disclosure relates to a dental composition, wherein the liquid carrier is present in a range of about 20% to about 60% with respect to weight of the dental composition.

The present disclosure relates to a dental composition, further including at least organic liquid having a concentration in a range of about 0.1% to about 50% with respect to weight of the dental composition.

The present disclosure relates to a dental composition, wherein the the active agent includes an aldehyde, an olefinic monomer, or a combination of the aldehyde and the olefinic monomer.

The present disclosure relates to a dental composition, wherein the aldehyde is selected from formaldehyde, propionaldehyde, butyraldehyde, glutaraldehyde, glycolaldehyde, benzaldehyde, or a combination thereof.

The present disclosure relates to a dental composition, wherein the olefinic monomer includes an ester of acrylic or methacrylic acid.

The present disclosure relates to a dental composition, wherein the active agent includes silver diamine fluoride. The present disclosure relates to a dental composition, wherein the active agent includes silver diamine fluoride in a range of from about 1% to about 65% with respect to weight of the dental composition. The present disclosure relates to a dental composition, wherein the active agent includes silver diamine fluoride in a range of from about 1% to about 60% with respect to weight of the dental composition. The present disclosure relates to a dental composition, wherein the active agent includes silver diamine fluoride in a range of from about 1% to about 55% with respect to weight of the dental composition. The present disclosure relates to a dental composition, wherein the active agent includes silver diamine fluoride in a range of from about 1% to about 50% with respect to weight of the dental composition.

The present disclosure relates to a dental composition, wherein the dental composition exhibits relatively enhanced handleability and storage stability compared to the dental composition without the thickening agent.

The present disclosure relates to a dental composition, wherein the thickening agent is present in an amount ranging from about 0.1 wt % to about 30 wt % of the composition. The present disclosure relates to a dental composition, wherein the thickening agent is present in an amount ranging from about 0.1 wt % to about 20 wt % of the composition. The present disclosure relates to a dental composition, wherein the thickening agent is present in an amount ranging from about 0.1 wt % to about 15 wt % of the composition. The present disclosure relates to a dental composition, wherein the thickening agent is present in an amount ranging from about 0.5 wt % to about 30 wt % of the composition. The present disclosure relates to a dental composition, wherein the thickening agent is present in an amount ranging from about 0.5 wt % to about 20 wt % of the composition. The present disclosure relates to a dental composition, wherein the thickening agent is present in an amount ranging from about 0.5 wt % to about 15 wt % of the composition.

The present disclosure relates to a dental composition, wherein the dental composition exhibits viscosity of from about 50 cP to about 10,000 cP. The present disclosure relates to a dental composition, wherein the dental composition exhibits viscosity of from about 50 cP to about 9,000 cP. The present disclosure relates to a dental composition, wherein the dental composition exhibits viscosity of from about 50 cP to about 8,000 cP. The present disclosure relates to a dental composition, wherein the dental composition exhibits viscosity of from about 50 cP to about 7,000 cP. The present disclosure relates to a dental composition, wherein the dental composition exhibits viscosity of from about 50 cP to about 5,000 cP. The present disclosure relates to a dental composition, wherein the dental composition exhibits viscosity of from about 50 cP to about 4,000 cP. The present disclosure relates to a dental composition, wherein the dental composition exhibits viscosity of from about 50 cP to about 3,000 cP. The present disclosure relates to a dental composition, wherein the dental composition exhibits viscosity of from about 50 cP to about 2,000 cP.

The present disclosure relates to a dental composition, wherein the active agent includes an anti-calculus agent, a remineralization agent, an antimicrobial agent, an antioxidant, an antiplaque agent, an anti-inflammatory agent, a desensitizing agent, and a combination thereof. The present disclosure relates to a dental composition, the dental composition is to be used as a pretreatment of a surface of tooth in dental restoration.

The present disclosure relates to a dental composition, further including HEMA.

The present disclosure relates to a dental composition, the amount of HEMA in the dental composition is from about 25 wt. % to about 45 wt. % with respect to the weight of the dental composition. The present disclosure relates to a dental composition, the amount of the polyvinylpyrrolidone present in the dental composition is from about 1 wt. % to about 12 wt. % with respect to the weight of the dental composition. The present disclosure relates to a dental composition, the amount of the polyvinylpyrrolidone present in the dental composition is from about 1 wt. % to about 8 wt. % with respect to the weight of the dental composition. The present disclosure relates to a dental composition, the amount of the polyvinylpyrrolidone present in the dental composition is from about 2 wt. % to about 12 wt. % with respect to the weight of the dental composition. The present disclosure relates to a dental composition, the amount of the polyvinylpyrrolidone present in the dental composition is from about 2 wt. % to about 10 wt. % with respect to the weight of the dental composition. The present disclosure relates to a dental composition, the amount of the polyvinylpyrrolidone present in the dental composition is from about 2 wt. % to about 8 wt. % with respect to weight of the dental composition.

The present disclosure relates to a dental composition, wherein the dental composition exhibits adhesion strength of the dental composition to dentin of at least about 10 MPa. The present disclosure relates to a dental composition, wherein the dental composition exhibits adhesion strength of the dental composition to dentin of at least about 15 MPa. The present disclosure relates to a dental composition, wherein the dental composition exhibits adhesion strength of the dental composition to dentin of at least about 20 MPa. The present disclosure relates to a dental composition, wherein the dental composition exhibits adhesion strength of the dental composition to dentin of at least about 25 MPa. The present disclosure relates to a dental composition, wherein the dental composition exhibits adhesion strength of the dental composition to dentin of at least about 26 MPa. The present disclosure relates to a dental composition, wherein the dental composition exhibits adhesion strength of the dental composition to dentin of at least about 27 MPa. The present disclosure relates to a dental composition, wherein the dental composition exhibits adhesion strength of the dental composition to dentin of at least about 28 MPa. The present disclosure relates to a dental composition, wherein the dental composition exhibits adhesion strength of the dental composition to dentin of at least about 29 MPa. The present disclosure relates to a dental composition, wherein the dental composition exhibits adhesion strength of the dental composition to dentin of at least about 29.5 MPa. The present disclosure relates to a dental composition, wherein the dental composition exhibits adhesion strength of the dental composition to dentin of at least about 30 MPa. The present disclosure relates to a dental composition, wherein the dental composition exhibits adhesion strength of the dental composition to dentin of at least about 30.5 MPa. The present disclosure relates to a dental composition, wherein the dental composition exhibits adhesion strength of the dental composition to dentin of at least about 31 MPa. The present disclosure relates to a dental composition, wherein the dental composition exhibits adhesion strength of the dental composition to dentin of at least about 31.5 MPa. The present disclosure relates to a dental composition, wherein the dental composition exhibits adhesion strength of the dental composition to dentin of at least about 32 MPa. The present disclosure relates to a dental composition, wherein the dental composition exhibits adhesion strength of the dental composition to dentin of at least about 32.5 MPa. The present disclosure relates to a dental composition, wherein the dental composition exhibits adhesion strength of the dental composition to dentin of at least about 33 MPa. The present disclosure relates to a dental composition, wherein the dental composition exhibits adhesion strength of the dental composition to dentin of at least about 33.5 MPa. The present disclosure relates to a dental composition, wherein the dental composition exhibits adhesion strength of the dental composition to dentin of at least about 34 MPa. The present disclosure relates to a dental composition, wherein the dental composition exhibits adhesion strength of the dental composition to dentin of at least about 35 MPa. The present disclosure relates to a dental composition, wherein the dental composition exhibits adhesion strength of the dental composition to dentin of from about 10 to about 35 MPa. The present disclosure relates to a dental composition, wherein the dental composition exhibits adhesion strength of the dental composition to dentin of from about 15 to about 35 MPa. The present disclosure relates to a dental composition, wherein the dental composition exhibits adhesion strength of the dental composition to dentin of from about 20 to about 35 MPa. The present disclosure relates to a dental composition, wherein the dental composition exhibits adhesion strength of the dental composition to dentin of from about 25 to about 35 MPa. The present disclosure relates to a dental composition, wherein the dental composition exhibits adhesion strength of the dental composition to dentin of from about 10 to about 40 MPa. The present disclosure relates to a dental composition, wherein the dental composition exhibits adhesion strength of the dental composition to dentin of from about 15 to about 40 MPa. The present disclosure relates to a dental composition, wherein the dental composition exhibits adhesion strength of the dental composition to dentin of from about 20 to about 40 MPa. The present disclosure relates to a dental composition, wherein the dental composition exhibits adhesion strength of the dental composition to dentin of from about 25 to about 40 MPa.

The present disclosure relates to a dental composition, wherein the dental composition substantially maintains viscosity or non-paste form after the dental composition is stored from about 42° C. to about 60° C. for a period of time.

The present disclosure relates to a dental composition, wherein the active agent includes a mineral acid selected from phosphoric acid, nitric acid, hydrochloric acid or sulfuric acid.

The present disclosure relates to a dental composition, wherein the active agent includes phosphoric acid.

The present disclosure relates to a dental composition, wherein the amount of the phosphoric acid in the dental composition is in a range of about 20% to about 80% with respect to weight of the dental composition. The present disclosure relates to a dental composition, wherein the amount of the phosphoric acid in the dental composition is 85% phosphoric acid in a range of about 20% to about 80% with respect to weight of the dental composition. The present disclosure relates to a dental composition, wherein the amount of the phosphoric acid in the dental composition is in a range of about 20% to about 70% with respect to weight of the dental composition. The present disclosure relates to a dental composition, wherein the amount of the phosphoric acid in the dental composition is 85% phosphoric acid in a range of about 20% to about 70% with respect to weight of the dental composition. The present disclosure relates to a dental composition, wherein the amount of the phosphoric acid in the dental composition is in a range of about 20% to about 65% with respect to weight of the dental composition. The present disclosure relates to a dental composition, wherein the amount of the phosphoric acid in the dental composition is 85% phosphoric acid in a range of about 20% to about 65% with respect to weight of the dental composition. The present disclosure relates to a dental composition, wherein the amount of the phosphoric acid in the dental composition is in a range of about 20% to about 50% with respect to weight of the dental composition. The present disclosure relates to a dental composition, wherein the amount of the phosphoric acid in the dental composition is 85% phosphoric acid in a range of about 20% to about 50% with respect to weight of the dental composition. The present disclosure relates to a dental composition, wherein the amount of the phosphoric acid in the dental composition is in a range of about 35% to about 50% with respect to weight of the dental composition. The present disclosure relates to a dental composition, wherein the amount of the phosphoric acid in the dental composition is 85% phosphoric acid in a range of about 35% to about 50% with respect to weight of the dental composition. The present disclosure relates to a dental composition, wherein the amount of the phosphoric acid in the dental composition is in a range of about 35% to about 45% with respect to weight of the dental composition. The present disclosure relates to a dental composition, wherein the amount of the phosphoric acid in the dental composition is 85% phosphoric acid in a range of about 35% to about 45% with respect to weight of the dental composition.

The present disclosure relates to a dental composition, the amount of the polyvinylpyrrolidone present in the dental composition is from about 1 wt. % to about 10 wt. % with respect to the weight of the dental composition. The present disclosure relates to a dental composition, the amount of the polyvinylpyrrolidone present in the dental composition is from about 1 wt. % to about 8 wt. % with respect to the weight of the dental composition. The present disclosure relates to a dental composition, the amount of the polyvinylpyrrolidone present in the dental composition is from about 1 wt. % to about 7 wt. % with respect to the weight of the dental composition. The present disclosure relates to a dental composition, the amount of the polyvinylpyrrolidone present in the dental composition is from about 2 wt. % to about 10 wt. % with respect to the weight of the dental composition. The present disclosure relates to a dental composition, the amount of the polyvinylpyrrolidone present in the dental composition is from about 2 wt. % to about 8 wt. % with respect to the weight of the dental composition. The present disclosure relates to a dental composition, the amount of the polyvinylpyrrolidone present in the dental composition is from about 2 wt. % to about 7 wt. % with respect to weight of the dental composition.

The present disclosure relates to a dental composition, the dental composition exhibits the viscosity of from about 50 cP to about 1500 cP. The present disclosure relates to a dental composition, the dental composition exhibits the viscosity of from about 50 cP to about 1400 cP. The present disclosure relates to a dental composition, the dental composition exhibits the viscosity of from about 50 cP to about 1300 cP. The present disclosure relates to a dental composition, the dental composition exhibits the viscosity of from about 50 cP to about 300 cP. The present disclosure relates to a dental composition, the dental composition exhibits the viscosity of from about 300 cP to about 800 cP. The present disclosure relates to a dental composition, the dental composition exhibits the viscosity of from about 350 cP to about 700 cP. The present disclosure relates to a dental composition, the dental composition exhibits the viscosity of from about 500 cP to about 1500 cP. The present disclosure relates to a dental composition, the dental composition exhibits the viscosity of from about 600 cP to about 1400 cP. The present disclosure relates to a dental composition, the dental composition exhibits the viscosity of from about 700 cP to about 1400 cP. The present disclosure relates to a dental composition, the dental composition exhibits the viscosity of from about 100 cP to about 1500 after the dental composition is stored at around 60° C. for about 8 weeks. The present disclosure relates to a dental composition, the dental composition exhibits the viscosity of from about 100 cP to about 1400 after the dental composition is stored at around 60° C. for about 8 weeks. The present disclosure relates to a dental composition, the dental composition exhibits the viscosity of from about 100 cP to about 1300 after the dental composition is stored at around 60° C. for about 8 weeks. The present disclosure relates to a dental composition, the dental composition exhibits the viscosity of from about 100 cP to about 300 after the dental composition is stored at around 60° C. for about 8 weeks. The present disclosure relates to a dental composition, the dental composition exhibits the viscosity of from about 350 cP to about 800 after the dental composition is stored at around 60° C. for about 8 weeks. The present disclosure relates to a dental composition, the dental composition exhibits the viscosity of from about 400 cP to about 700 after the dental composition is stored at around 60° C. for about 8 weeks. The present disclosure relates to a dental composition, the dental composition exhibits the viscosity of from about 550 cP to about 1500 after the dental composition is stored at around 60° C. for about 8 weeks. The present disclosure relates to a dental composition, the dental composition exhibits the viscosity of from about 650 cP to about 1400 after the dental composition is stored at around 60° C. for about 8 weeks. The present disclosure relates to a dental composition, the dental composition exhibits the viscosity of from about 800 cP to about 1400 after the dental composition is stored at around 60° C. for about 8 weeks. The present disclosure relates to a dental composition, the viscosity of the dental composition does not increase or increases less than about 1000 cP after the dental composition is stored at around 60° C. for about 8 weeks. The present disclosure relates to a dental composition, the viscosity of the dental composition does not increase or increases less than about 700 cP after the dental composition is stored at around 60° C. for about 8 weeks. The present disclosure relates to a dental composition, the viscosity of the dental composition does not increase or increases less than about 500 cP after the dental composition is stored at around 60° C. for about 8 weeks. The present disclosure relates to a dental composition, the viscosity of the dental composition does not increase or increases less than about 400 cP after the dental composition is stored at around 60° C. for about 8 weeks.

The present disclosure relates to a dental composition, the pH of the dental composition is substantially maintained, or decreases, or does not increase after the dental composition is stored at around 60° C. for about 4 weeks. The present disclosure relates to a dental composition, the pH of the dental composition decreases by about 0.05 or more and less than about 2 after the dental composition is stored at around 60° C. for about 4 weeks. The present disclosure relates to a dental composition, the pH of the dental composition decreases by about 0.1 or more and less than about 2 after the dental composition is stored at around 60° C. for about 4 weeks. The present disclosure relates to a dental composition, the pH of the dental composition decreases by about 0.05 or more and less than about 1 after the dental composition is stored at around 60° C. for about 4 weeks. The present disclosure relates to a dental composition, the pH of the dental composition decreases by about 0.1 or more and less than about 1 after the dental composition is stored at around 60° C. for about 4 weeks. The present disclosure relates to a dental composition, the pH of the dental composition decreases by about 0.05 or more and less than about 0.5 after the dental composition is stored at around 60° C. for about 4 weeks. The present disclosure relates to a dental composition, the pH of the dental composition decreases by about 0.1 or more and less than about 0.5 after the dental composition is stored at around 60° C. for about 4 weeks.

The present disclosure relates to a method including applying dental composition to a tooth surface.

The present disclosure relates to a device to apply the dental composition to a tooth surface, including: a container configured to contain the dental composition.

### DETAILED DESCRIPTION

It is to be understood that the disclosure is not limited to the embodiments of the present disclosure described below, as variations of the embodiments may be made and still fall within the scope of the appended claims. It is also to be understood that the terminology employed is for the purpose of describing embodiments and is not intended to be limiting.

As used herein, the singular forms “a,” “an” and “the” include plural reference unless the context clearly dictates otherwise. Unless defined otherwise, all technical and scientific terms used herein have the same meaning as commonly understood to one of ordinary skill in the art to which this disclosure belongs.

As used herein, the term “oral cavity” refers to the mouth, which includes the lips, the lining inside the cheeks and lips, the front two thirds of the tongue, the upper and lower gums, the floor of the mouth under the tongue, the bony roof the mouth, and the small area behind the wisdom teeth.

As used herein, the term “extreme condition” refers to an aqueous condition with a high pH (pH above about 12), a low pH (pH below about 2), or high concentration of salts (over 20 wt % of salts).

As used herein, the terms “runny” and “runniness” refer to the tendency of a dental composition to drip from dispensing tips or run off of tooth area rather than staying where placed.

As used herein, the term “syneresis” refers to extraction of a liquid from gel/paste due to gel/paste contraction.

Oral hygiene plays a pivotal role in oral health and the prevention of oral disease. Poor oral hygiene can result in the accumulation of plaque. Plaque is a diverse biofilm that develops as bacterial preferentially attach to surfaces. Ultimately, an oral environment shift occurs, resulting in tooth caries, gingival inflammation, etc. Therefore, oral hygiene needs to be practiced daily using a toothpaste and mechanical toothbrushing in order to prevent plaque accumulation.

The present disclosure relates to dental compositions, e.g., aqueous dental compositions, and methods of using these dental compositions in treatment in restorations, more particularly disinfection compositions for better bonding of restoration to tooth tissues, and etching compositions used in etching teeth.

In some embodiments, teeth restoration of tooth structures is often accomplished by a sequential application of at least one among a cleaner, a primer, an etchant, an adhesive, followed by a dental restorative material to a prepared tooth cavity. In some embodiments, these pretreatment processes based on the sequential application before restorative material filling can be used to promote the bonding and longevity of restorations. In some embodiments, caries finder can be used to identify caries-affected tissues, especially caries-affected dentin, to be removed as these tissues can compromise the longevity of bonding.

In some embodiments, dental etchants are materials used in restorative dentistry to prepare tooth surfaces for bonding procedures. In some embodiments, dental etching compositions can be used to remove the smear layer and demineralize enamel, dentin or both so a restorative composition or orthodontic bracket can better adhere to the prepared tooth structure. In some embodiments, etchants have an extremely low pH (pH is usually below 1). In some embodiments, when they contacted gum line, it can cause irritation, burning, inflammation, etc. In some embodiments, in the selective enamel etching technique used in restoration, etching enamel without dentin is preferred. See, e.g., [Perdigao J, Arujo E, Adhesive dentistry: current concepts and clinical considerations, J Esthet Restor Dent, 2020:1-18.].

In some embodiments, various types of etchants and various compounds exhibiting a pH level to function as an etchant can be used. In some embodiments, an organic or inorganic acid is used as an etchant to etch hard tissues. In some embodiments, phosphoric acid as an etchant can be used. In some embodiments, phosphoric acid etchant can be used. at a concentration of 20-50%. In some embodiments, phosphoric acid etchant can be used. at a concentration of 30-40%. In some embodiments, phosphoric acid etchant can be used. at a concentration of 35-37%. In some embodiments, when applied to enamel and dentin, the etchant can create microscopic roughness on the tooth surface by partially dissolving minerals, which increases the surface area and allows for better adhesion of bonding agents and restorative materials. In some embodiments, the etching process can be for achieving a relatively strong, long-lasting bond between the tooth and the restoration. In some embodiments, proper technique and controlling parameters can be important, as over-etching can lead to demineralization of the tooth structure and reduced bond strength. In some embodiments, the etching time can be controlled and thoroughly rinse the etchant to ensure optimal results.

In some embodiments, it has been unexpectedly found that viscosity of the etchant can be controlled to control the residual time. In some embodiments, it has been unexpectedly found that a range of viscosity of the etchant can result in controlled or increased residual time as the etchant can stay in the applied surface area and reduce the damage to surrounding tissues. In some embodiments, viscosity of the etchant and concentration can be controlled to control the residual time. In some embodiments, increasing viscosity of the etchant can increase residual and application time of the etchant, which can allow a lower concentration of etchant for the etchant application and reduce possible damage to surrounding tissue or oral cavity.

In some embodiments, the viscosity of the etchant can be adjusted to regulate its residual time on a targeted surface. By modifying the viscosity, the etchant can be retained within the intended application area for a longer period, allowing for a more controlled etching process while minimizing unintended spread to adjacent regions. In some embodiments, it has been unexpectedly found that a specific range of viscosity for the etchant can result in an optimized residual time, ensuring that the etchant remains on the desired surface long enough to achieve effective etching without excessively diffusing into surrounding tissues. This controlled retention can significantly reduce unintended damage to neighboring structures, such as soft tissue or other sensitive areas in the oral cavity, thereby enhancing safety and precision in its application.

In some embodiments, both the viscosity and concentration of the etchant can be adjusted in a coordinated manner to regulate the residual time effectively. By relatively more fine-tuning these parameters, it is possible to achieve a balance that provides sufficient etching efficacy while mitigating potential adverse effects.

In some embodiments, increasing the viscosity of the etchant has been found to extend both the residual time and the overall application time. This prolonged retention allows for the use of a lower concentration of the etchant while maintaining its effectiveness. As a result, the risk of unintended damage to surrounding tissues, including soft tissue and the broader oral cavity, can be significantly reduced. This approach offers an improved safety profile while preserving the efficacy of the etching process, making it particularly advantageous in applications where precision and tissue preservation are critical.

In some embodiments, the etching time can be controlled based on the viscosity of the disinfecting agent. In some embodiments, viscosity of the etchant can be controlled to control the residual time. In some embodiments, viscosity of the etchant and concentration can be controlled to control the residual time. In some embodiments, the application process involves applying the etchant with increased viscosity according to the present disclosure for from about 3 seconds to about 7 minutes. In some embodiments, the application process involves applying the etchant for from about 5 seconds to about 5 minutes. In some embodiments, the application process involves applying the etchant for from about 5 seconds to about 4 minutes. In some embodiments, the application process involves applying the etchant for from about 5 seconds to about 2 minutes. In some embodiments, the application process involves applying the etchant for from about 5 seconds to about 1 minutes.

In some embodiments, dental disinfectants can be used in preparing enamel surfaces for bonding procedures, e.g., serving to eliminate bacteria and contaminants that could compromise adhesion. In some embodiments, various types of disinfectants can be used. For example, in some embodiments, disinfectants may include chlorhexidine, sodium hypochlorite, and hydrogen peroxide. In some embodiments, the application process involves applying the disinfectant for a period of time, before applying the bonding agent. In some embodiments, proper technique and controlling parameters for disinfecting process can be important.

In some embodiments, it has been unexpectedly discovered that the viscosity of a dental disinfectant can be adjusted to regulate its residual time on an oral surface. By modifying the viscosity, the disinfectant can be retained in the intended application area for a longer duration, ensuring prolonged contact with the targeted surfaces and enhancing its antimicrobial efficacy while minimizing unintended spread to surrounding tissues.

In some embodiments, it has been unexpectedly found that maintaining the dental disinfectant within a specific viscosity range can optimize its residual time, allowing for sufficient exposure to effectively eliminate or reduce microbial load while preventing excessive runoff or dilution by saliva. This controlled retention ensures that the disinfectant remains in place long enough to achieve thorough disinfection while reducing unintended exposure to adjacent soft tissues, such as the gums, cheeks, or tongue, which could otherwise experience irritation or discomfort.

In some embodiments, both the viscosity and concentration of the dental disinfectant can be adjusted in a coordinated manner to regulate the residual time effectively. By fine-tuning these parameters, it is possible to achieve a balance that maximizes disinfection efficacy while minimizing potential adverse effects, such as mucosal irritation or undesirable residue formation.

In some embodiments, increasing the viscosity of the dental disinfectant has been found to extend both its residual time and overall application time. This extended retention allows for the use of a lower concentration of active disinfecting agents while maintaining effective antimicrobial performance. As a result, the risk of unintended tissue irritation, excessive exposure, or rapid dissipation due to salivary flow can be significantly reduced. This approach provides an improved safety profile while ensuring that the disinfectant remains effective in controlling oral pathogens, making it particularly beneficial in dental treatments where prolonged antimicrobial action is desired.

In some embodiments, the disinfecting time can be controlled based on the viscosity of the disinfecting agent. In some embodiments, viscosity of the disinfectant can be controlled to control the residual time. In some embodiments, viscosity of the disinfectant and concentration can be controlled to control the residual time. In some embodiments, the application process involves applying the disinfectant with increased viscosity according to the present disclosure for from about 5 seconds to about 5 minutes. In some embodiments, the application process involves applying the disinfectant for from about 5 seconds to about 4 minutes. In some embodiments, the application process involves applying the disinfectant for from about 5 seconds to about 3 minutes. In some embodiments, the application process involves applying the disinfectant for from about 10 seconds to about 2 minutes. In some embodiments, the application process involves applying the disinfectant for from about 10 seconds to about 1 minutes.

In some embodiments, glutaraldehyde, silver diamine fluoride, or a combination thereof can be used to disinfect the prepared tooth cavity and increase, facilitate, and/or improve the bonding between the tooth structure and the overlying adhesive. See, e.g., [Baba N., et al, Effect of disinfectants containing glutaraldehyde on bonding of a tri-n-butylborane initiated resin to dentine, 2002, 29(5): 478-483]. In some embodiments, in the case of preparing the tooth for a crown or a deep cavity that is to be filled with a restorative composition, hemostatic composition may be needed to stop or slow bleeding of the exposed pulp.

In some embodiments, caries can be identified using caries identifiers. In some embodiments, caries finders can contain negatively charged dyes. In some embodiments, when exposed to demineralized enamel and dentin, they formed protonated groups, such as carboxylic acid (COOH) and/or protonated free amines (NH₃⁺) which covalently and/or electrostatically bond to the carious tissues.

For example, in some embodiments, glutaraldehyde can inactivate MMP enzymes. It is a cross-linking reagent capable of bonding to amine groups of proteins. It can form covalent bonds between two proteins forming highly cross-linked insoluble protein aggregates. In some embodiments, through that way, it can act as disinfectant in treating tooth cavity. Moreover, in some embodiments, the protein aggregates in dentin tubules can form plugs which can seal the dentin tubules, reduce permeability of fluid and in turn the incidence of dentinal hypersensitivity. Therefore, in some embodiments, glutaraldehyde can be used to treat tooth cavity before restoration after acid-etching. In some embodiments, silver diamine fluoride (SDF) can generally be used as a 38% solution for treating caries and dentine hypersensitivity. In some embodiments, it is a colorless alkaline solution with a pH value between 9 and 10. In some embodiments, SDF can inhibit biofilm formation and promote remineralization, counteract demineralization, prevent collagen and occlude dentinal tubules. In some embodiments, SDF therapy can cause permanent black staining on enamel and dentin. Its stain on the skin often lasts for a few weeks. In some embodiments, although it does not cause any pain, stains on patient's lips, chin or face or the operator's hands can be unaesthetic.

Currently, these treatment agents, i.e., caries finders, etchants, disinfectants, are low viscosity liquids that often spread across the treated surface and can be hard to control where the liquid travels. In some embodiments, this can be problematic and causes patient discomfort and neighboring tissue damages. For example, when applied to a tooth, it can advance to undesired areas or neighboring tissue or gum and irritate or damage the tissue or gum. Therefore, there is an unmet need for dental liquid composition to be thickened to have better application control, e.g., for a controlled spreading. Moreover, there is a further need for a dental liquid composition to be storage stable. Moreover, one of the technical solutions provided according to the present disclosure is relatively more precise control of liquids being applied such as highly reactive or physically or chemically active liquids such as a cleaning agent, an etching agent, a disinfecting agent, a bonding agent, etc., e.g., in terms of physical localization and residual time. In some embodiments, viscosity or flowability of the highly reactive or physically or chemically active liquids, along with types of chemicals, concentrations, etc., can be increased or controlled to increase or control the physical localization and residual time.

For the dental liquid composition with highly reactive or physically or chemically active properties, e.g., extremely acidic, alkaline, as well as excessive salts conditions, it has been a technical problem and challenge that these agents are difficult to be thickened or mixed with other agents to increase its viscosity. Moreover, many thickening agents or agents to increase viscosity can be toxic to living organism, tissue human, or patient. Accordingly, in some embodiments, medically acceptable compositions or compositions adequate or safe, to be used in oral or other medical applications with a practicality, especially with increased viscosity while maintaining the flowability or storage stability or with a thickened property, especially when the compositions exhibit highly reactive or physically or chemically active properties, such as extremely acidic, alkaline, or excessive salts conditions, have not been available in the field and non-existent. For example, they were thickened by inorganic thickeners, such as fumed silica. However, inorganic thickener thickened compositions tend to be bulky and quickly dried out during storage, resulting in hard dispensing. For example, compositions based on inorganic thickeners can also have a syneresis issue, i.e., liquid is excluded from the gel due to gel contraction during storage. The leached liquid in these compositions need to be pre-dispensed before clinical application. Some Polymeric thickeners have been used for thickening. However, typical organic thickeners tend to precipitate, be tacky, coagulate or otherwise quickly been degraded in the highly reactive or physically or chemically active environment, such as extremely acidic, alkaline or a high electrolyte environment.

The present disclosure is related to a dental composition that is used to promote adhesion and longevity of dental restoration. The present disclosure is related to a dental composition comprising a liquid carrier comprising water, at least one organic liquid such as alcohols, polyols, olefinic monomer, glycerin, propylene glycol, polyethylene glycol, ethanol, acetone and a combination thereof, a thickening agent comprising a polyvinylpyrrolidone having vinylpyrrolidone side groups, and an active agent present in the composition.

The present disclosure relates to dental compositions composed of a liquid carrier, a polymeric thickener and an active dental agent that is stable in aggressive conditions, such as extremely low or low pH (pH<about 1, pH<about 1.3, pH<about 1.5, pH<about 1.8, pH<about 2, pH<about 2.2, pH<about 2.5, pH<about 2.8, pH<about 3.0, pH<about 3.5, pH<about 4. pH<about 5), extremely high or high pH (pH>about 13, pH>about 12.8, pH>about 12.5, pH>about 12.3, pH>about 12, pH>about 11.8, pH>about 11.5, pH>about 11.3, pH>about 11, pH>about 10.7, pH>about 10.5, pH>about 10.3, pH>about 10, pH>about 9.8), high concentration of salts or cross-linkers.

Surprisingly, it was unexpectedly found that the compositions according to the present disclosure, e.g., by increasing thickness or increasing viscosity, exhibited a longer effective shelf life (i.e., the dental compositions remain homogeneous longer) than they would using other currently available thickening compositions for the systems and application. The dental compositions of current disclosure exhibit consistent homogeneity without being stringy or coagulation after prolonged storage. The invented dental composition provide improved ease of application, including better flowability easily dispensed from syringe tips, readily dispersible, and easily rinsed off. In some embodiments, the composition viscosity or thickness was controlled to increase the shelf life.

Accordingly, longer shelf life can be measured based on the consistent homogeneity. In some embodiments, after 6 months, the homogeneity will maintain at least about 80% of the consistency of the initial specification. In some embodiments, after 6 months, the homogeneity will maintain at least about 85% of the consistency of the initial specification. In some embodiments, after 6 months, the homogeneity will maintain at least about 90% of the consistency of the initial specification. In some embodiments, after 6 months, the homogeneity will maintain at least about 95% of the consistency of the initial specification. In some embodiments, after 6 months, the homogeneity will maintain at least about 97% of the consistency of the initial specification. In some embodiments, after 6 months, the homogeneity will maintain at least about 99% of the consistency of the initial specification.

Accordingly, longer shelf life can be measured based on the maintained viscosity of the initial specification. In some embodiments, after 6 months, at least about 80% of the consistency of the initial viscosity will be maintained. In some embodiments, after 6 months, at least about 85% of the consistency of the initial viscosity will be maintained. In some embodiments, after 6 months, at least about 90% of the consistency of the initial viscosity will be maintained. In some embodiments, after 6 months, at least about 93% of the consistency of the initial viscosity will be maintained. In some embodiments, after 6 months, at least about 95% of the consistency of the initial viscosity will be maintained. In some embodiments, after 6 months, at least about 97% of the consistency of the initial viscosity will be maintained. In some embodiments, after 6 months, at least about 99% of the consistency of the initial viscosity will be maintained.

In some embodiments, the at least one thickening agent may present in amount ranging from about 0.1 wt % to about 35 wt % of the composition. In some embodiments, the at least one thickening agent may present in amount ranging from about 0.1 wt % to about 32 wt % of the composition. In some embodiments, the at least one thickening agent may present in amount ranging from about 0.1 wt % to about 30 wt % of the composition. In some embodiments, the at least one thickening agent may present in amount ranging from about 0.1 wt % to about 28 wt % of the composition. In some embodiments, the at least one thickening agent may present in amount ranging from about 0.1 wt % to about 25 wt % of the composition. In some embodiments, the at least one thickening agent may present in amount ranging from about 0.1 wt % to about 22 wt % of the composition. In some embodiments, the at least one thickening agent may present in amount ranging from about 0.1 wt % to about 20 wt % of the composition. In some embodiments, the at least one thickening agent may present in amount ranging from about 0.1 wt % to about 18 wt % of the composition. In some embodiments, the at least one thickening agent may present in amount ranging from about 0.1 wt % to about 15 wt % of the composition. In some embodiments, the at least one thickening agent may present in amount ranging from about 1 wt % to about 35 wt % of the composition. In some embodiments, the at least one thickening agent may present in amount ranging from about 1 wt % to about 32 wt % of the composition. In some embodiments, the at least one thickening agent may present in amount ranging from about 1 wt % to about 30 wt % of the composition. In some embodiments, the at least one thickening agent may present in amount ranging from about 1 wt % to about 28 wt % of the composition. In some embodiments, the at least one thickening agent may present in amount ranging from about 1 wt % to about 25 wt % of the composition. In some embodiments, the at least one thickening agent may present in amount ranging from about 1 wt % to about 22 wt % of the composition. In some embodiments, the at least one thickening agent may present in amount ranging from about 1 wt % to about 20 wt % of the composition. In some embodiments, the at least one thickening agent may present in amount ranging from about 1 wt % to about 18 wt % of the composition. In some embodiments, the at least one thickening agent may present in amount ranging from about 1 wt % to about 15 wt % of the composition. In some embodiments, the at least one thickening agent may present in amount ranging from about 3 wt % to about 35 wt % of the composition. In some embodiments, the at least one thickening agent may present in amount ranging from about 3 wt % to about 32 wt % of the composition. In some embodiments, the at least one thickening agent may present in amount ranging from about 3 wt % to about 30 wt % of the composition. In some embodiments, the at least one thickening agent may present in amount ranging from about 3 wt % to about 28 wt % of the composition. In some embodiments, the at least one thickening agent may present in amount ranging from about 3 wt % to about 25 wt % of the composition. In some embodiments, the at least one thickening agent may present in amount ranging from about 3 wt % to about 22 wt % of the composition. In some embodiments, the at least one thickening agent may present in amount ranging from about 3 wt % to about 20 wt % of the composition. In some embodiments, the at least one thickening agent may present in amount ranging from about 3 wt % to about 18 wt % of the composition. In some embodiments, the at least one thickening agent may present in amount ranging from about 3 wt % to about 15 wt % of the composition. In some embodiments, the at least one thickening agent may present in amount ranging from about 5 wt % to about 35 wt % of the composition. In some embodiments, the at least one thickening agent may present in amount ranging from about 5 wt % to about 32 wt % of the composition. In some embodiments, the at least one thickening agent may present in amount ranging from about 5 wt % to about 30 wt % of the composition. In some embodiments, the at least one thickening agent may present in amount ranging from about 5 wt % to about 28 wt % of the composition. In some embodiments, the at least one thickening agent may present in amount ranging from about 5 wt % to about 25 wt % of the composition. In some embodiments, the at least one thickening agent may present in amount ranging from about 5 wt % to about 22 wt % of the composition. In some embodiments, the at least one thickening agent may present in amount ranging from about 5 wt % to about 20 wt % of the composition. In some embodiments, the at least one thickening agent may present in amount ranging from about 5 wt % to about 18 wt % of the composition. In some embodiments, the at least one thickening agent may present in amount ranging from about 5 wt % to about 15 wt % of the composition.

In some embodiments, the active agent may present in the composition in an amount ranging from about 0.1 wt % to about 65 wt % of the composition. In some embodiments, the active agent may present in the composition in an amount ranging from about 0.1 wt % to about 60 wt % of the composition. In some embodiments, the active agent may present in the composition in an amount ranging from about 0.1 wt % to about 55 wt % of the composition. In some embodiments, the active agent may present in the composition in an amount ranging from about 0.1 wt % to about 50 wt % of the composition. In some embodiments, the active agent may present in the composition in an amount ranging from about 0.1 wt % to about 40 wt % of the composition. In some embodiments, the active agent may present in the composition in an amount ranging from about 0.1 wt % to about 35 wt % of the composition. In some embodiments, the active agent may present in the composition in an amount ranging from about 0.1 wt % to about 30 wt % of the composition. In some embodiments, the active agent may present in the composition in an amount ranging from about 5 wt % to about 65 wt % of the composition. In some embodiments, the active agent may present in the composition in an amount ranging from about 5 wt % to about 60 wt % of the composition. In some embodiments, the active agent may present in the composition in an amount ranging from about 5 wt % to about 55 wt % of the composition. In some embodiments, the active agent may present in the composition in an amount ranging from about 5 wt % to about 50 wt % of the composition. In some embodiments, the active agent may present in the composition in an amount ranging from about 5 wt % to about 40 wt % of the composition. In some embodiments, the active agent may present in the composition in an amount ranging from about 5 wt % to about 35 wt % of the composition. In some embodiments, the active agent may present in the composition in an amount ranging from about 5 wt % to about 30 wt % of the composition. In some embodiments, the active agent may present in the composition in an amount ranging from about 10 wt % to about 65 wt % of the composition. In some embodiments, the active agent may present in the composition in an amount ranging from about 10 wt % to about 60 wt % of the composition. In some embodiments, the active agent may present in the composition in an amount ranging from about 10 wt % to about 55 wt % of the composition. In some embodiments, the active agent may present in the composition in an amount ranging from about 10 wt % to about 50 wt % of the composition. In some embodiments, the active agent may present in the composition in an amount ranging from about 10 wt % to about 40 wt % of the composition. In some embodiments, the active agent may present in the composition in an amount ranging from about 10 wt % to about 35 wt % of the composition. In some embodiments, the active agent may present in the composition in an amount ranging from about 10 wt % to about 30 wt % of the composition. In some embodiments, the active agent may present in the composition in an amount ranging from about 15 wt % to about 65 wt % of the composition. In some embodiments, the active agent may present in the composition in an amount ranging from about 15 wt % to about 60 wt % of the composition. In some embodiments, the active agent may present in the composition in an amount ranging from about 15 wt % to about 55 wt % of the composition. In some embodiments, the active agent may present in the composition in an amount ranging from about 15 wt % to about 50 wt % of the composition. In some embodiments, the active agent may present in the composition in an amount ranging from about 15 wt % to about 40 wt % of the composition. In some embodiments, the active agent may present in the composition in an amount ranging from about 15 wt % to about 35 wt % of the composition. In some embodiments, the active agent may present in the composition in an amount ranging from about 15 wt % to about 30 wt % of the composition.

In some embodiments, the the active agent comprises an aldehyde, an olefinic monomer, or a combination of aldehyde and olefinic monomer. In some embodiments, the aldehyde is selected from formaldehyde, propionaldehyde, butyraldehyde, glutaraldehyde, glycolaldehyde and benzaldehyde. In some embodiments, the olefinic monomer is the esters of acrylic or methacrylic acid. In some embodiments, the active agent comprises a mineral acid selected from phosphoric acid, nitric acid, hydrochloric acid or sulfuric acid. In some embodiments, the mineral acid is phosphoric acid.

In some embodiments, the phosphoric acid is present in the composition in an amount ranging from about 10% to about 70% by weight. In some embodiments, the phosphoric acid is present in the composition in an amount ranging from about 10% to about 65% by weight. In some embodiments, the phosphoric acid is present in the composition in an amount ranging from about 10% to about 60% by weight. In some embodiments, the phosphoric acid is present in the composition in an amount ranging from about 10% to about 55% by weight. In some embodiments, the phosphoric acid is present in the composition in an amount ranging from about 10% to about 50% by weight. In some embodiments, the phosphoric acid is present in the composition in an amount ranging from about 10% to about 45% by weight.

In some embodiments, the phosphoric acid is present in the composition in an amount ranging from about 20% to about 70% by weight. In some embodiments, the phosphoric acid is present in the composition in an amount ranging from about 20% to about 65% by weight. In some embodiments, the phosphoric acid is present in the composition in an amount ranging from about 20% to about 60% by weight. In some embodiments, the phosphoric acid is present in the composition in an amount ranging from about 20% to about 55% by weight. In some embodiments, the phosphoric acid is present in the composition in an amount ranging from about 20% to about 50% by weight. In some embodiments, the phosphoric acid is present in the composition in an amount ranging from about 20% to about 45% by weight. In some embodiments, the phosphoric acid is present in the composition in an amount ranging from about 30% to about 70% by weight. In some embodiments, the phosphoric acid is present in the composition in an amount ranging from about 30% to about 65% by weight. In some embodiments, the phosphoric acid is present in the composition in an amount ranging from about 30% to about 60% by weight. In some embodiments, the phosphoric acid is present in the composition in an amount ranging from about 30% to about 55% by weight. In some embodiments, the phosphoric acid is present in the composition in an amount ranging from about 30% to about 50% by weight. In some embodiments, the phosphoric acid is present in the composition in an amount ranging from about 30% to about 45% by weight. In some embodiments, the phosphoric acid is present in the composition in an amount ranging from about 40% to about 70% by weight. In some embodiments, the phosphoric acid is present in the composition in an amount ranging from about 40% to about 65% by weight. In some embodiments, the phosphoric acid is present in the composition in an amount ranging from about 40% to about 60% by weight. In some embodiments, the phosphoric acid is present in the composition in an amount ranging from about 40% to about 55% by weight. In some embodiments, the phosphoric acid is present in the composition in an amount ranging from about 40% to about 50% by weight. In some embodiments, the phosphoric acid is present in the composition in an amount ranging from about 40% to about 45% by weight.

In some embodiments, the active agent can comprise silver diamine fluoride.

In some embodiments, the silver diamine fluoride is present in the composition in an amount ranging from about 1% to about 70% by weight. In some embodiments, the silver diamine fluoride is present in the composition in an amount ranging from about 1% to about 65% by weight. In some embodiments, the silver diamine fluoride is present in the composition in an amount ranging from about 1% to about 60% by weight. In some embodiments, the silver diamine fluoride is present in the composition in an amount ranging from about 1% to about 55% by weight. In some embodiments, the silver diamine fluoride is present in the composition in an amount ranging from about 1% to about 50% by weight. In some embodiments, the silver diamine fluoride is present in the composition in an amount ranging from about 1% to about 45% by weight. In some embodiments, the silver diamine fluoride is present in the composition in an amount ranging from about 3% to about 70% by weight. In some embodiments, the silver diamine fluoride is present in the composition in an amount ranging from about 3% to about 65% by weight. In some embodiments, the silver diamine fluoride is present in the composition in an amount ranging from about 3% to about 60% by weight. In some embodiments, the silver diamine fluoride is present in the composition in an amount ranging from about 3% to about 55% by weight. In some embodiments, the silver diamine fluoride is present in the composition in an amount ranging from about 3% to about 50% by weight. In some embodiments, the silver diamine fluoride is present in the composition in an amount ranging from about 3% to about 45% by weight. In some embodiments, the silver diamine fluoride is present in the composition in an amount ranging from about 5% to about 70% by weight. In some embodiments, the silver diamine fluoride is present in the composition in an amount ranging from about 5% to about 65% by weight. In some embodiments, the silver diamine fluoride is present in the composition in an amount ranging from about 5% to about 60% by weight. In some embodiments, the silver diamine fluoride is present in the composition in an amount ranging from about 5% to about 55% by weight. In some embodiments, the silver diamine fluoride is present in the composition in an amount ranging from about 5% to about 50% by weight. In some embodiments, the silver diamine fluoride is present in the composition in an amount ranging from about 5% to about 45% by weight.

In some embodiments, the active agent may comprise an anti-calculus agent, a remineralization agent, an antimicrobial agent, an antioxidant, an antiplaque agent, an anti-inflammatory agent, a desensitizing agent, and a combination thereof.

In some embodiments, the dental composition is capable of forming a homogeneous gel and maintaining homogeneousness, texture, viscosity and/or consistency for an extended period of time.

In some embodiments, the polyvinylpyrrolidone comprises a homopolymer or a copolymer of one or more monomers having one or more vinylpyrrolidone groups.

In some embodiments, the liquid carrier can a compound selected from the group consisting of water, alcohols, glycolpolyols, glycerin, olefinic monomers and a mixture thereof.

In some embodiments, the liquid carrier having an amount in a range of from about 10% to about 70% by weight. In some embodiments, the liquid carrier having an amount in a range of from about 10% to about 60% by weight. In some embodiments, the liquid carrier having an amount in a range of from about 10% to about 50% by weight. In some embodiments, the liquid carrier having an amount in a range of from about 10% to about 40% by weight. In some embodiments, the liquid carrier having an amount in a range of from about 10% to about 30% by weight. In some embodiments, the liquid carrier having an amount in a range of from about 20% to about 70% by weight. In some embodiments, the liquid carrier having an amount in a range of from about 20% to about 60% by weight. In some embodiments, the liquid carrier having an amount in a range of from about 20% to about 50% by weight. In some embodiments, the liquid carrier having an amount in a range of from about 20% to about 40% by weight. In some embodiments, the liquid carrier having an amount in a range of from about 20% to about 30% by weight. In some embodiments, the liquid carrier having an amount in a range of from about 30% to about 70% by weight. In some embodiments, the liquid carrier having an amount in a range of from about 30% to about 60% by weight. In some embodiments, the liquid carrier having an amount in a range of from about 30% to about 50% by weight. In some embodiments, the liquid carrier having an amount in a range of from about 30% to about 40% by weight. In some embodiments, the liquid carrier having an amount in a range of about 30% by weight.

In some embodiments, the at least one organic liquid may present in an amount ranging from about 0.1 wt % to about 35 wt % of the composition. In some embodiments, the at least one organic liquid may present in an amount ranging from about 0.1 wt % to about 32 wt % of the composition. In some embodiments, the at least one organic liquid may present in an amount ranging from about 0.1 wt % to about 30 wt % of the composition. In some embodiments, the at least one organic liquid may present in an amount ranging from about 0.1 wt % to about 28 wt % of the composition. In some embodiments, the at least one organic liquid may present in an amount ranging from about 0.1 wt % to about 25 wt % of the composition. In some embodiments, the at least one organic liquid may present in an amount ranging from about 0.1 wt % to about 22 wt % of the composition. In some embodiments, the at least one organic liquid may present in an amount ranging from about 0.1 wt % to about 20 wt % of the composition. In some embodiments, the at least one organic liquid may present in an amount ranging from about 0.1 wt % to about 18 wt % of the composition. In some embodiments, the at least one organic liquid may present in an amount ranging from about 0.1 wt % to about 15 wt % of the composition. In some embodiments, the at least one organic liquid may present in an amount ranging from about 1 wt % to about 35 wt % of the composition. In some embodiments, the at least one organic liquid may present in an amount ranging from about 1 wt % to about 32 wt % of the composition. In some embodiments, the at least one organic liquid may present in an amount ranging from about 1 wt % to about 30 wt % of the composition. In some embodiments, the at least one organic liquid may present in an amount ranging from about 1 wt % to about 28 wt % of the composition. In some embodiments, the at least one organic liquid may present in an amount ranging from about 1 wt % to about 25 wt % of the composition. In some embodiments, the at least one organic liquid may present in an amount ranging from about 1 wt % to about 22 wt % of the composition. In some embodiments, the at least one organic liquid may present in an amount ranging from about 1 wt % to about 20 wt % of the composition. In some embodiments, the at least one organic liquid may present in an amount ranging from about 1 wt % to about 18 wt % of the composition. In some embodiments, the at least one organic liquid may present in an amount ranging from about 1 wt % to about 15 wt % of the composition. In some embodiments, the at least one organic liquid may present in an amount ranging from about 3 wt % to about 35 wt % of the composition. In some embodiments, the at least one organic liquid may present in an amount ranging from about 3 wt % to about 32 wt % of the composition. In some embodiments, the at least one organic liquid may present in an amount ranging from about 3 wt % to about 30 wt % of the composition. In some embodiments, the at least one organic liquid may present in an amount ranging from about 3 wt % to about 28 wt % of the composition. In some embodiments, the at least one organic liquid may present in an amount ranging from about 3 wt % to about 25 wt % of the composition. In some embodiments, the at least one organic liquid may present in an amount ranging from about 3 wt % to about 22 wt % of the composition. In some embodiments, the at least one organic liquid may present in an amount ranging from about 3 wt % to about 20 wt % of the composition. In some embodiments, the at least one organic liquid may present in an amount ranging from about 3 wt % to about 18 wt % of the composition. In some embodiments, the at least one organic liquid may present in an amount ranging from about 3 wt % to about 15 wt % of the composition. In some embodiments, the at least one organic liquid may present in an amount ranging from about 5 wt % to about 35 wt % of the composition. In some embodiments, the at least one organic liquid may present in an amount ranging from about 5 wt % to about 32 wt % of the composition. In some embodiments, the at least one organic liquid may present in an amount ranging from about 5 wt % to about 30 wt % of the composition. In some embodiments, the at least one organic liquid may present in an amount ranging from about 5 wt % to about 28 wt % of the composition. In some embodiments, the at least one organic liquid may present in an amount ranging from about 5 wt % to about 25 wt % of the composition. In some embodiments, the at least one organic liquid may present in an amount ranging from about 5 wt % to about 22 wt % of the composition. In some embodiments, the at least one organic liquid may present in an amount ranging from about 5 wt % to about 20 wt % of the composition. In some embodiments, the at least one organic liquid may present in an amount ranging from about 5 wt % to about 18 wt % of the composition. In some embodiments, the at least one organic liquid may present in an amount ranging from about 5 wt % to about 15 wt % of the composition.

The present disclosure is directed to controlling of the viscosity so as to better control the residual time and residual area of the composition on a surface over a period of time. In some embodiments, the composition directed to the present disclosure exhibits enhanced handleability and storage stability. In some embodiments, the composition is a gel.

In some embodiments, the composition exhibits a viscosity of from about 50 cP to about 30,000 cP. In some embodiments, the composition exhibits a viscosity of from about 50 cP to about 27,000 cP. In some embodiments, the composition exhibits a viscosity of from about 50 cP to about 25,000 cP. In some embodiments, the composition exhibits a viscosity of from about 50 cP to about 23,000 cP. In some embodiments, the composition exhibits a viscosity of from about 50 cP to about 20,000 cP. In some embodiments, the composition exhibits a viscosity of from about 50 cP to about 18,000 cP. In some embodiments, the composition exhibits a viscosity of from about 50 cP to about 17,000 cP. In some embodiments, the composition exhibits a viscosity of from about 50 cP to about 15,000 cP. In some embodiments, the composition exhibits a viscosity of from about 50 cP to about 13,000 cP. In some embodiments, the composition exhibits a viscosity of from about 50 cP to about 12,000 cP. In some embodiments, the composition exhibits a viscosity of from about 50 cP to about 11,000 cP. In some embodiments, the composition exhibits a viscosity of from about 50 cP to about 10,000 cP. In some embodiments, the composition exhibits a viscosity of from about 50 cP to about 9,000 cP. In some embodiments, the composition exhibits a viscosity of from about 50 cP to about 8,000 cP. In some embodiments, the composition exhibits a viscosity of from about 50 cP to about 7,000 cP. In some embodiments, the composition exhibits a viscosity of from about 50 cP to about 6,000 cP. In some embodiments, the composition exhibits a viscosity of from about 50 cP to about 5,000 cP. In some embodiments, the composition exhibits a viscosity of from about 50 cP to about 3,000 cP. In some embodiments, the composition exhibits a viscosity of from about 50 cP to about 2,000 cP. In some embodiments, the composition exhibits a viscosity of from about 50 cP to about 1,500 cP.

In some embodiments, the composition exhibits a viscosity of from about 100 cP to about 30,000 cP. In some embodiments, the composition exhibits a viscosity of from about 100 cP to about 27,000 cP. In some embodiments, the composition exhibits a viscosity of from about 100 cP to about 25,000 cP. In some embodiments, the composition exhibits a viscosity of from about 100 cP to about 23,000 cP. In some embodiments, the composition exhibits a viscosity of from about 100 cP to about 20,000 cP. In some embodiments, the composition exhibits a viscosity of from about 100 cP to about 18,000 cP. In some embodiments, the composition exhibits a viscosity of from about 100 cP to about 17,000 cP. In some embodiments, the composition exhibits a viscosity of from about 100 cP to about 15,000 cP. In some embodiments, the composition exhibits a viscosity of from about 100 cP to about 13,000 cP. In some embodiments, the composition exhibits a viscosity of from about 100 cP to about 12,000 cP. In some embodiments, the composition exhibits a viscosity of from about 100 cP to about 11,000 cP. In some embodiments, the composition exhibits a viscosity of from about 100 cP to about 10,000 cP. In some embodiments, the composition exhibits a viscosity of from about 100 cP to about 9,000 cP. In some embodiments, the composition exhibits a viscosity of from about 100 cP to about 8,000 cP. In some embodiments, the composition exhibits a viscosity of from about 100 cP to about 7,000 cP. In some embodiments, the composition exhibits a viscosity of from about 100 cP to about 6,000 cP. In some embodiments, the composition exhibits a viscosity of from about 100 cP to about 5,000 cP. In some embodiments, the composition exhibits a viscosity of from about 100 cP to about 3,000 cP. In some embodiments, the composition exhibits a viscosity of from about 100 cP to about 2,000 cP. In some embodiments, the composition exhibits a viscosity of from about 100 cP to about 1,500 cP.

In some embodiments, the composition exhibits a viscosity of from about 200 cP to about 30,000 cP. In some embodiments, the composition exhibits a viscosity of from about 200 cP to about 27,000 cP. In some embodiments, the composition exhibits a viscosity of from about 200 cP to about 25,000 cP. In some embodiments, the composition exhibits a viscosity of from about 200 cP to about 23,000 cP. In some embodiments, the composition exhibits a viscosity of from about 200 cP to about 20,000 cP. In some embodiments, the composition exhibits a viscosity of from about 200 cP to about 18,000 cP. In some embodiments, the composition exhibits a viscosity of from about 200 cP to about 17,000 cP. In some embodiments, the composition exhibits a viscosity of from about 200 cP to about 15,000 cP. In some embodiments, the composition exhibits a viscosity of from about 200 cP to about 13,000 cP. In some embodiments, the composition exhibits a viscosity of from about 200 cP to about 12,000 cP. In some embodiments, the composition exhibits a viscosity of from about 200 cP to about 11,000 cP. In some embodiments, the composition exhibits a viscosity of from about 200 cP to about 10,000 cP. In some embodiments, the composition exhibits a viscosity of from about 200 cP to about 9,000 cP. In some embodiments, the composition exhibits a viscosity of from about 200 cP to about 8,000 cP. In some embodiments, the composition exhibits a viscosity of from about 200 cP to about 7,000 cP. In some embodiments, the composition exhibits a viscosity of from about 200 cP to about 6,000 cP. In some embodiments, the composition exhibits a viscosity of from about 200 cP to about 5,000 cP. In some embodiments, the composition exhibits a viscosity of from about 200 cP to about 3,000 cP. In some embodiments, the composition exhibits a viscosity of from about 200 cP to about 2,000 cP. In some embodiments, the composition exhibits a viscosity of from about 200 cP to about 1,500 cP.

In some embodiments, the composition exhibits a viscosity of from about 500 cP to about 30,000 cP. In some embodiments, the composition exhibits a viscosity of from about 500 cP to about 27,000 cP. In some embodiments, the composition exhibits a viscosity of from about 500 cP to about 25,000 cP. In some embodiments, the composition exhibits a viscosity of from about 500 cP to about 23,000 cP. In some embodiments, the composition exhibits a viscosity of from about 500 cP to about 20,000 cP. In some embodiments, the composition exhibits a viscosity of from about 500 cP to about 18,000 cP. In some embodiments, the composition exhibits a viscosity of from about 500 cP to about 17,000 cP. In some embodiments, the composition exhibits a viscosity of from about 500 cP to about 15,000 cP. In some embodiments, the composition exhibits a viscosity of from about 500 cP to about 13,000 cP. In some embodiments, the composition exhibits a viscosity of from about 500 cP to about 12,000 cP. In some embodiments, the composition exhibits a viscosity of from about 500 cP to about 11,000 cP. In some embodiments, the composition exhibits a viscosity of from about 500 cP to about 10,000 cP. In some embodiments, the composition exhibits a viscosity of from about 500 cP to about 9,000 cP. In some embodiments, the composition exhibits a viscosity of from about 500 cP to about 8,000 cP. In some embodiments, the composition exhibits a viscosity of from about 500 cP to about 7,000 cP. In some embodiments, the composition exhibits a viscosity of from about 500 cP to about 6,000 cP. In some embodiments, the composition exhibits a viscosity of from about 500 cP to about 5,000 cP. In some embodiments, the composition exhibits a viscosity of from about 500 cP to about 3,000 cP. In some embodiments, the composition exhibits a viscosity of from about 500 cP to about 2,000 cP. In some embodiments, the composition exhibits a viscosity of from about 500 cP to about 1,500 cP.

In some embodiments, the composition exhibits a viscosity of from about 1000 cP to about 30,000 cP. In some embodiments, the composition exhibits a viscosity of from about 1000 cP to about 27,000 cP. In some embodiments, the composition exhibits a viscosity of from about 1000 cP to about 25,000 cP. In some embodiments, the composition exhibits a viscosity of from about 1000 cP to about 23,000 cP. In some embodiments, the composition exhibits a viscosity of from about 1000 cP to about 20,000 cP. In some embodiments, the composition exhibits a viscosity of from about 1000 cP to about 18,000 cP. In some embodiments, the composition exhibits a viscosity of from about 1000 cP to about 17,000 cP. In some embodiments, the composition exhibits a viscosity of from about 1000 cP to about 15,000 cP. In some embodiments, the composition exhibits a viscosity of from about 1000 cP to about 13,000 cP. In some embodiments, the composition exhibits a viscosity of from about 1000 cP to about 12,000 cP. In some embodiments, the composition exhibits a viscosity of from about 1000 cP to about 11,000 cP. In some embodiments, the composition exhibits a viscosity of from about 1000 cP to about 10,000 cP. In some embodiments, the composition exhibits a viscosity of from about 1000 cP to about 9,000 cP. In some embodiments, the composition exhibits a viscosity of from about 1000 cP to about 8,000 cP. In some embodiments, the composition exhibits a viscosity of from about 1000 cP to about 7,000 cP. In some embodiments, the composition exhibits a viscosity of from about 1000 cP to about 6,000 cP. In some embodiments, the composition exhibits a viscosity of from about 1000 cP to about 5,000 cP. In some embodiments, the composition exhibits a viscosity of from about 1000 cP to about 3,000 cP. In some embodiments, the composition exhibits a viscosity of from about 1000 cP to about 2,000 cP. In some embodiments, the composition exhibits a viscosity of from about 1000 cP to about 1,500 cP.

In some embodiments, the composition exhibits a viscosity of from about 2000 cP to about 30,000 cP. In some embodiments, the composition exhibits a viscosity of from about 2000 cP to about 27,000 cP. In some embodiments, the composition exhibits a viscosity of from about 2000 cP to about 25,000 cP. In some embodiments, the composition exhibits a viscosity of from about 2000 cP to about 23,000 cP. In some embodiments, the composition exhibits a viscosity of from about 2000 cP to about 20,000 cP. In some embodiments, the composition exhibits a viscosity of from about 2000 cP to about 18,000 cP. In some embodiments, the composition exhibits a viscosity of from about 2000 cP to about 17,000 cP. In some embodiments, the composition exhibits a viscosity of from about 2000 cP to about 15,000 cP. In some embodiments, the composition exhibits a viscosity of from about 2000 cP to about 13,000 cP. In some embodiments, the composition exhibits a viscosity of from about 2000 cP to about 12,000 cP. In some embodiments, the composition exhibits a viscosity of from about 2000 cP to about 11,000 cP. In some embodiments, the composition exhibits a viscosity of from about 2000 cP to about 10,000 cP. In some embodiments, the composition exhibits a viscosity of from about 2000 cP to about 9,000 cP. In some embodiments, the composition exhibits a viscosity of from about 2000 cP to about 8,000 cP. In some embodiments, the composition exhibits a viscosity of from about 2000 cP to about 7,000 cP. In some embodiments, the composition exhibits a viscosity of from about 2000 cP to about 6,000 cP. In some embodiments, the composition exhibits a viscosity of from about 2000 cP to about 5,000 cP. In some embodiments, the composition exhibits a viscosity of from about 2000 cP to about 3,000 cP.

In some embodiments, the composition exhibits a viscosity of from about 3000 cP to about 30,000 cP. In some embodiments, the composition exhibits a viscosity of from about 3000 cP to about 27,000 cP. In some embodiments, the composition exhibits a viscosity of from about 3000 cP to about 25,000 cP. In some embodiments, the composition exhibits a viscosity of from about 3000 cP to about 23,000 cP. In some embodiments, the composition exhibits a viscosity of from about 3000 cP to about 20,000 cP. In some embodiments, the composition exhibits a viscosity of from about 3000 cP to about 18,000 cP. In some embodiments, the composition exhibits a viscosity of from about 3000 cP to about 17,000 cP. In some embodiments, the composition exhibits a viscosity of from about 3000 cP to about 15,000 cP. In some embodiments, the composition exhibits a viscosity of from about 3000 cP to about 13,000 cP. In some embodiments, the composition exhibits a viscosity of from about 3000 cP to about 12,000 cP. In some embodiments, the composition exhibits a viscosity of from about 3000 cP to about 11,000 cP. In some embodiments, the composition exhibits a viscosity of from about 3000 cP to about 10,000 cP. In some embodiments, the composition exhibits a viscosity of from about 3000 cP to about 9,000 cP. In some embodiments, the composition exhibits a viscosity of from about 3000 cP to about 8,000 cP. In some embodiments, the composition exhibits a viscosity of from about 3000 cP to about 7,000 cP. In some embodiments, the composition exhibits a viscosity of from about 3000 cP to about 6,000 cP. In some embodiments, the composition exhibits a viscosity of from about 3000 cP to about 5,000 cP.

In some embodiments, the composition exhibits a viscosity of from about 5000 cP to about 30,000 cP. In some embodiments, the composition exhibits a viscosity of from about 5000 cP to about 27,000 cP. In some embodiments, the composition exhibits a viscosity of from about 5000 cP to about 25,000 cP. In some embodiments, the composition exhibits a viscosity of from about 5000 cP to about 23,000 cP. In some embodiments, the composition exhibits a viscosity of from about 5000 cP to about 20,000 cP. In some embodiments, the composition exhibits a viscosity of from about 5000 cP to about 18,000 cP. In some embodiments, the composition exhibits a viscosity of from about 5000 cP to about 17,000 cP. In some embodiments, the composition exhibits a viscosity of from about 5000 cP to about 15,000 cP. In some embodiments, the composition exhibits a viscosity of from about 5000 cP to about 13,000 cP. In some embodiments, the composition exhibits a viscosity of from about 5000 cP to about 12,000 cP. In some embodiments, the composition exhibits a viscosity of from about 5000 cP to about 11,000 cP. In some embodiments, the composition exhibits a viscosity of from about 5000 cP to about 10,000 cP. In some embodiments, the composition exhibits a viscosity of from about 5000 cP to about 9,000 cP. In some embodiments, the composition exhibits a viscosity of from about 5000 cP to about 8,000 cP. In some embodiments, the composition exhibits a viscosity of from about 5000 cP to about 7,000 cP. In some embodiments, the composition exhibits a viscosity of from about 5000 cP to about 6,000 cP. In some embodiments, the composition exhibits a viscosity of about 5000 cP.

In some embodiments, the composition exhibits a viscosity of from about 7000 cP to about 30,000 cP. In some embodiments, the composition exhibits a viscosity of from about 7000 cP to about 27,000 cP. In some embodiments, the composition exhibits a viscosity of from about 7000 cP to about 25,000 cP. In some embodiments, the composition exhibits a viscosity of from about 7000 cP to about 23,000 cP. In some embodiments, the composition exhibits a viscosity of from about 7000 cP to about 20,000 cP. In some embodiments, the composition exhibits a viscosity of from about 7000 cP to about 18,000 cP. In some embodiments, the composition exhibits a viscosity of from about 7000 cP to about 17,000 cP. In some embodiments, the composition exhibits a viscosity of from about 7000 cP to about 15,000 cP. In some embodiments, the composition exhibits a viscosity of from about 7000 cP to about 13,000 cP. In some embodiments, the composition exhibits a viscosity of from about 7000 cP to about 12,000 cP. In some embodiments, the composition exhibits a viscosity of from about 7000 cP to about 11,000 cP. In some embodiments, the composition exhibits a viscosity of from about 7000 cP to about 10,000 cP. In some embodiments, the composition exhibits a viscosity of from about 7000 cP to about 9,000 cP. In some embodiments, the composition exhibits a viscosity of from about 7000 cP to about 8,000 cP. In some embodiments, the composition exhibits a viscosity of about 7000 cP. In some embodiments, the composition exhibits a viscosity of about 6000 cP. In some embodiments, the composition exhibits a viscosity of about 5000 cP. In some embodiments, the composition exhibits a viscosity of about 4000 cP. In some embodiments, the composition exhibits a viscosity of about 3000 cP. In some embodiments, the composition exhibits a viscosity of about 2000 cP. In some embodiments, the composition exhibits a viscosity of about 1500 cP. In some embodiments, the composition exhibits a viscosity of about 1000 cP. In some embodiments, the composition exhibits a viscosity of about 500 cP.

In some embodiments, the composition is used as a pretreatment in dental restorations.

The present disclosure relates to a method of providing the composition to a patient's tooth surface.

The present disclosure relates to dental compositions composed of a liquid carrier, a polymeric thickener and an active dental agent that is stable in aggressive conditions, such as extremely low pH (pH<1), extremely high pH (pH>13), high concentration of salts or cross-linkers. Surprisingly, it was unexpectedly found that the invented dental compositions exhibit a longer effective shelf life (i.e., the dental compositions remain homogeneous longer) than they would use other thickening systems. The dental compositions of current disclosure exhibit consistent homogeneity without being stringy or coagulation after prolonged storage. In some embodiments, the dental composition related to the present disclosure provides improved ease of application, including easily dispensed from syringe tips, readily dispersible, and easily rinsed off.

In some embodiments, the present disclosure provides an aqueous dental treatment composition that includes: 0.1 wt % to 10 wt % of polymeric thickener; 0.1 wt % to 50 wt % of active agent; at least 20 wt % of water; and at least 1% of organic liquid such as glycol, alcohol or olefinic monomers, wherein the weight percentages are based on the total weight of the aqueous composition. In some embodiments, the present disclosure provide a polymeric thickener. The polymeric thickening agents preferably are polyvinylpyrrolidone (PVPs) comprises a homopolymer or a copolymer of one or more monomers having one or more vinylpyrrolidone groups. This thickener provides unique thickening effects in extreme conditions that are essentially stable, i.e. maintain rheological thickening properties even with prolonged storage.

In some embodiments, the liquid carrier can comprise water, and at least an organic solvent such as glycerin, propylene glycol, polyethylene glycol, ethanol, acetone or olefinic monomers. Surprisingly, it was found the present disclosed compositions which comprising both water and at least one organic liquid are storage stable in extremely conditions. They are easily dispensed from syringe tips, readily dispersible, and easily rinsed off They provide consistent smooth gel without being stringy or coagulation even after prolonged storage.

In some embodiments, the present disclosure can provide a thixotropic property, i.e. shear thinning when dispersed through a needle or agitated after being placed and then thickened when stopped agitation. This property not only facilitates gel dispensing from syringes but also improves application control of the dental compositions when applied on tooth tissue. It helps the thickened composition to be remained in place where applied. The present dental compositions do not have syneresis issue with prolonged storage and therefore avoid dispenser clog or liquid leaching issues that often occurred in silica-thickened compositions.

In some embodiments, the present disclosure provides a method of application of dental compositions to a patient's tooth surface. The method involves applying an aqueous dental treatment composition as disclosed herein to the patient's tooth surface.

In another embodiment, the present disclosure provides a method of treating prepared tooth cavity before restoration. The method involves applying an aqueous dental composition as disclosed herein to the patient's tooth surface.

### Thickening Agent

In some embodiments, the compositions of the present disclosure can also comprise one or more thickening agents in order to yield a composition with unexpectedly higher viscosity. In some embodiments, the polymeric thickening agent serves to increase the viscosity of the restoration pretreatment composition. In addition, the polymeric thickening agent can also serve to improve the handling properties of the composition, e.g., readily dispensable, smooth application, no stringy, no tacky, etc.

A particulate rheology-modifying agent is included within the compositions of the present disclosure to improve the rheological properties of the compositions. According to one embodiment, the rheology-modifying agent is included to offset the stringiness imparted by thickener and also to reduce or eliminate runniness. Rheology-modifying agents include insoluble hydrophilic polymers such as hydrophilic polyvinylpyrrolidone particulates commercially available as Kollidon and Luvicross, marketed by BASF, Mount Olive, N.J., USA, and POLYPLASDONE™ crospovidone polymer, marketed by Ashland Corporation, Wayne, N.J., USA. Other examples are modified celluloses such as cross-linked sodium carboxymethylcelluloses available as AC-DI-SOL®, PRIMELLOSE®, PHARMACEL XL®, EXPLOCEL®, and NYMCEL ZSX®, microcrystalline cellulose available as AVICEL®, PHARMACEL®, EMCOCELL®, VIVAPUR®, natural starches, such as maize starch, potato starch etc., modified starches such as carboxymethyl starches and sodium starch glycolate which are available as PRIMOJEL®, EXPLOTAB®, EXPLOSOL®, and starch derivatives such as amylose. In the present disclosure, each of them may be used solely or two or more thereof may be used jointly. The rheology-modifying agents may be included in a broad range from about 1% to about 25% by weight of the composition. A preferred mean particle size of the rheology-modifying agent is in the range of 3 to 10 m. The mean particle size can be determined using a Malvern Mastersizer™. For the rheology-modifying agent, at least 90% of the particles by weight preferable have a size below 15 μm.

In some embodiments, the thickening agent may comprise an n-vinyl heterocyclic polymer. In some embodiments, the n-vinyl heterocyclic polymer is derived from an n-heterocyclic vinyl monomer, which may comprise n-vinyl heterocyclic monomers having from 3 to 7 atoms in a heterocyclic ring, including a carbonyl carbon atom and a nitrogen heteroatom containing a vinyl group. In some embodiments, the ring contains 5 or 6 atoms, comprises heteroatoms such as oxygen or sulfur, and may be substituted or unsubstituted.

In some embodiments, the thickening agent may contain poly-N-vinyl polylactams, In some embodiments, the agent containing poly-N-vinyl polylactams may include but are not limited to poly-N-vinyl pyrrolidone, poly-N-vinyl-2-piperidone, poly-N-vinyl-2-caprolactam, poly-N-vinyl-3-methyl-2-caprolactam, poly-N-vinyl-3-methyl-2-piperidone, poly-N-vinyl-4-methyl-2-pyrrolidone, and mixtures thereof. In some embodiments, the polymer can be selected from the group consisting of poly-N-vinyl poly-2-pyrrolidone, poly-N-vinyl-poly-2-piperidone, poly-N-vinyl-2-caprolactam and mixtures thereof.

In some embodiments, PVP refers to a polymer containing vinylpyrrolidone as a monomeric unit. In some embodiments, the monomeric unit consists of a polar imide group, four non-polar methylene groups and a non-polar methane group. In some embodiments, the polymers include soluble and insoluble homopolymeric PVPs. In some embodiments, the polymeric thickener can be poly-N-vinyl-poly-2-pyrrolidone. In some embodiments, the poly-N-vinyl-poly-2-pyrrolidone is known as polyvinylpyrrolidone (“PVP”). In some embodiments, copolymers containing PVP include vinylpyrrolidone/vinyl acetate and vinyl pyrrolidone/dimethylamino-ethylmethacrylate may be used.

In some embodiments, soluble PVP polymers may include Povidone, Polyvidone, poly(N-vinyl-2-pyrrolidinone), poly(N-vinylbutyrolactam) and poly(1-vinyl-2-pyrrolidone). In some embodiments, these PVP polymers are not cross-linked.

For example, Polyvinylpyrrolidone (PVP), also commonly called polyvidone or povidone, is a water-soluble polymer made from the monomer N-vinylpyrrolidone. PVP is available in a range of molecular weights and related viscosities and can be selected according to the desired application properties. PVP has properties that can be controlled based on its molecular weight and polymerization method. For example, the rate of dissolution is inversely proportional to the molecular weight whereas the viscosity, complexing nature, and adhesive power are directly proportional to the molecular weight of PVP. Even the biological elimination of PVP upon parenteral administration is also inversely proportional to its molecular weight. Hence, a specific molecular weight PVP that is suitable for the desired application can be selected. By controlling the properties of the PVP, the adhesion properties such as adhesion duration can be controlled to achieve the cleaning ability of the cleaning composition 20.

In some embodiments, a strongly swellable crosslinked PVP homopolymer may be used. In some embodiments, a strongly swellable crosslinked PVP may include but not limited to FLEXITHIX™ PVP polymer, ACP-1120, ACP-1179, and ACP-1180 available from (Ashland Specialty (Wayne, NJ). Disclosures of these polymers are provided in the following publications, each of which is hereby incorporated in its entirety by reference: U.S. Pat. Nos. 5,073,614; 5,312,619; 5,139,770; 5,716,634; 5,470,884; 5,759,524; 5,997,887; 6,024,942; international PCT application PCT/US10/26973; international PCT application PCT/US10/26976; international PCT application PCT/US10/26940; international PCT application PCT/US 11/32993; and international PCT application PCT/US 11/34515.

In some embodiments, a swellable but water-insoluble cross-linked PVP homopolymer may be used. In some embodiments, such polymers include those commercially available as Kollidon and Luvicross, marketed by BASF (Mount Olive, NJ); Polyplasdone, marketed by Ashland (Wayne, NJ).

It is understood that embodiments disclosed herein are not limited to a PVP homopolymer or its copolymer of a specific molecular weight and that any equivalent PVP of acceptable purity, such as pharmaceutical grade, is within a scope of embodiments disclosed herein.

In some embodiments, the composition comprises from about 0.01% to about 30% PVP polymeric thickener. In some embodiments, the composition comprises from about 0.01% to about 20% PVP. In some embodiments, the composition comprises from about 0.01% to about 15% PVP. In some embodiments, the composition comprises from about 0.01% to about 10% PVP In some embodiments, the composition comprises about 1% to about 20% PVP polymer. In some embodiments, the composition comprises about 1% to about 15% PVP polymer. In some embodiments, the composition comprises about 1% to about 10% PVP polymer. In some embodiments, the composition comprises about 1% to about 8% PVP polymer. In some embodiments, the composition comprises about 1% to about 7% PVP polymer. In some embodiments, the composition comprises about 1% to about 5% PVP polymer. For example, in some embodiments, the composition comprises about 9% PVP polymer. For example, in some embodiments, the composition comprises about 8% PVP polymer. For example, in some embodiments, the composition comprises about 7% PVP polymer. For example, in some embodiments, the composition comprises about 7% PVP polymer. For example, in some embodiments, the composition comprises about 6% PVP polymer. For example, in some embodiments, the composition comprises about 5% PVP polymer. For example, in some embodiments, the composition comprises about 4% PVP polymer. In some embodiments, the composition comprises about 3% PVP polymer.

For example, in some embodiments, the composition comprises from about 0.01% to about 10% PVP polymeric thickener. In some embodiments, the composition comprises about 1% to about 5% PVP polymer. For example, in some embodiments, the composition comprises about 7% PVP polymer. In some embodiments, the composition comprises about 3% PVP polymer.

According to an aspect of the disclosure, an added abrasive agent can be eliminated and absence of the cleaning effect of an abrasive agent can be supplemented by a rheological effect, also defined herein as “lightweight abrasive” effect, or “rubbing” effect caused by shear force or viscous force.

According to an aspect of the disclosure, an unexpected synergetic effect of combining two different forms of PVPs was found and applied to formulate a composition, such as better controlling of rheology, viscosity, surface localization, etc. For example, in some embodiments, a water soluble PVP and a cross-linked PVP that is relatively less water-soluble or relatively not as water-soluble or relatively not water-soluble were combined together. It was unexpectedly found that, because both a water-soluble PVP and a cross-linked PVP share some molecular structural similarity, both PVPs were relatively more compatible to each other and relatively more miscible. Possibly due to the interaction of the cross-linked PVP that is not as water-soluble, the adhesive retention of the cleaning composition was unexpectedly and substantially increased when both a water-soluble PVP and a cross-linked PVP were included, compared to compositions containing a water-soluble PVP without a cross-linked PVP and compositions containing a cross-linked PVP without a water-soluble PVP. In some embodiments, adding a water-soluble PVP to a cross-linked PVP unexpectedly increased spreadability or dispersibility of the cross-linked PVP over a hard-to-reach surface and over a surface curvature of a dental appliance. In some embodiments, because of the compatibility between a water-soluble PVP and a cross-linked PVP, combining a water-soluble PVP and a cross-linked PVP unexpectedly allowed more diverse and more precise design of a property of the composition.

In some embodiments, one or more polymeric thickeners may also be included in the present disclosure. In some embodiments, examples include but are not limited to STABILEZE™ RAPITHIX™ A-60, RAPITHIX™ A-100, ULTRATHIX™ P-100, and LUBRAJEL™ from Ashland Specialty (Wayne, NJ), etc.

In some embodiments, inorganic fillers may be added as a rheology-modifying agent in small amount, e.g., so as to reduce or eliminate runniness, or yield a coherent mass with little or no stringiness. In some embodiments, examples of inorganic fillers may include but not limited to colloidal silica, fumed silica, ground silica, aluminum oxide and titanium dioxide. In some embodiments, the composition comprises about 0.01% to about 10% fumed silica, such as fumed silica (Aerosil® 200), etc.

### Liquid Carrier

In some embodiments, the different types of compositions disclosed or related to present disclosure can comprise from about 1% to about 99%, from about 1% to about 99%, from about 1% to about 95%, from about 1% to about 90%, from about 1% to about 85%, from about 1% to about 80%, 5% to about 99%, from about 5% to about 99%, from about 5% to about 95%, from about 5% to about 90%, from about 5% to about 85%, from about 5% to about 80%, In some embodiments, the different types of compositions disclosed or related to present disclosure can comprise from about 10% to about 90%, by weight of the composition of a liquid carrier, e.g., water or other liquid agents. In some embodiments, the liquid carrier can thus comprise water, or a combination of water and one or more water soluble or dispensable liquid. For example, in some embodiments, liquid carriers comprising water, alcohol, various polyols and olefinic monomer are preferred.

In some embodiments, polyols can be added to the compositions in order to control the surface tension, or, e.g., to improve wettability. In some embodiments, examples of polyols may include glycerin, propylene glycol, polyethylene glycol, polypropylene glycol, etc. For example, in some embodiments, two or more different polyols can be mixed together to have related properties. In some embodiments, when a polyol is included within the compositions according to the present disclosure, polyol (e.g., one or more polyols) is included in an amount in a range of from about 0.1% to about 20% by weight of the composition. In some embodiments, when a polyol is included within the compositions according to the present disclosure, polyol (e.g., one or more polyols) is included in an amount in a range of from about 0.1% to about 15% by weight of the composition. For example, in some embodiments, when a polyol is included within the compositions according to the present disclosure, polyol (e.g., one or more polyols) is included in an amount in a range of from about 1% to about 10% by weight.

In some embodiments, the dental compositions according to or related to the present disclosure may include an olefinic monomer. In some embodiments, examples of suitable olefinic monomers include methacrylate (MMA), bisphenol A-glycidyl methacrylate (Bis-GMA), triethylene glycol dimethacrylate (TEGDMA), Diurethane dimethacrylate (UDMA), 2-hydroxyethyl methacrylate (HEMA), 2-hydroxyethyl methacrylate phosphate (HEMA-P), 10-methacryloyloxydecyl dihydrogen phosphate (10-MDP). Preferred monomers are HEMA, HEMA-P and 10-MDP, etc.

### Active Agent-Acid

In some embodiments, an active agent can be an acid. In some embodiments, the acid for use in the composition of current disclosure can be selected from a variety of acids that are able to etch teeth. In some embodiments, examples of dental etching agents include, but are not limited to mineral acids, such as phosphoric acid, boric acid, nitric acid, hydrochloric acid, and sulfuric acid, and organic acids, such as maleic acid, oxalic acid, acetic acid, citric acid, ethylenediaminetetraacetic acid and salts, and mixtures thereof. For example, in some embodiments, the acid can be phosphoric acid.

In some embodiments, according to the composition of current disclosure, the acid etching agent is in an amount so as to effectively etch dentin and enamel. In some embodiments, the acid etching agent is preferably included within the dental etching composition in an amount in a range of from about 10% to about 80% by weight of the etching composition. In some embodiments, the acid etching agent is preferably included within the dental etching composition in an amount in a range of from about 10% to about 70% by weight of the etching composition.

In some embodiments, according to the composition of current disclosure, the acid etching agent is in an amount so as to effectively etch dentin and enamel. In some embodiments, the acid etching agent is preferably included within the dental etching composition in an amount in a range of from about 10% to about 60% by weight of the etching composition. In some embodiments, the acid etching agent is preferably included within the dental etching composition in an amount in a range of from about 20% to about 50% by weight. In some embodiments, the acid etching agent is preferably included within the dental etching composition in an amount in a range of from about 30% to about 40% by weight.

In some embodiments, the present disclosure also provides methods of using the etchant compositions. For example, in some embodiments, a method of usage comprises first etching the hard tissues with the composition of the present disclosure, rinsing off and then applying a dental material, such as bonding agent, cement, restorative materials, sealant, dental primer, desensitizer, etc. In some embodiments, the hard tissues that can be etched refer to human and mammalian tissues, including bone, enamel, dentin and cementum. In some embodiments, in particular, the present disclosed compositions are used to etch enamel and dentin. In some embodiments, before the compositions application, the hard tissue is preferably cleaned using traditional methods, i.e., by brushing it with toothpaste, by polishing it with prophy paste, or by abrading it with a bur. In some embodiments, it should be thoroughly rinsed with water and air dried. In some embodiments, during etching, the disclosed compositions preferably remained on the treated surface for at least about 5 seconds to about 15 minutes. In some embodiments, during etching, the disclosed compositions preferably remained on the treated surface for at least about 5 seconds to about 10 minutes. In some embodiments, during etching, the disclosed compositions preferably remained on the treated surface for at least about 5 seconds to about 5 minutes. In some embodiments, during etching, the disclosed compositions preferably remained on the treated surface for at least about 5 seconds to about 3 minutes. In some embodiments, during etching, the disclosed compositions preferably remained on the treated surface for at least about 5 seconds to about 2 minutes. In some embodiments, more preferably, the etch compositions are allowed to remain on the treated surface for about 10 s to about 30 seconds. In some embodiments, shorter or longer etching time can also be used to provide the desired degree of etching and etching rate or concentration of etchant. In some embodiments, after etching, the etching compositions should be removed by water rinsing and dried before a dental material application.

### Active Agent-Disinfectants

In some embodiments, as a disinfectant, various types of compounds can be included as a disinfectant.

For example, in some embodiments, an active agent of the compositions according to the current disclosure can include an aldehyde. In some embodiments, the aldehyde that can be used in the disclosed composition can be any low molecular weight aliphatic mono or dialdehydes, preferably aliphatic aldehyde with 1 to 10 carbon atoms. In some embodiments, examples of aldehydes include formaldehyde, propionaldehyde, butyraldehyde, glutaraldehyde, glycolaldehyde and benzaldehyde it is preferred to contain 3 to 15 wt % of aldehyde.

In some embodiments, a dental composition of present disclosure can also include silver diamine fluoride as the disinfection agent. In some embodiments, a treatment composition includes at least 1.0 wt % of the composition. In some embodiments, a treatment composition include up to 50 wt % of the total composition. In some embodiments,
In some embodiments, a dental composition of present disclosure can also include sodium hydroxide, potassium hydroxide, a combination of sodium hydroxide and potassium hydroxide, etc. In some embodiments, in some embodiments, a treatment composition includes at least 1.0 wt % of the composition. In some embodiments, a treatment composition include up to 10 wt % of the total composition.

In some embodiments, a dental composition of present disclosure can also include chlorhexidine as the disinfection agent. In some embodiments, a treatment composition includes at least 0.1 wt % of the composition. In some embodiments, a treatment composition include up to 5 wt % of the total composition. In some embodiments,
In some embodiments, the present disclosure also provides methods of using dental compositions containing disinfectants. In some embodiments, a preferred method of usage comprises applying the composition of the disclosure to the hard tissues before or after acid etching, rinsing off and then applying a dental material, such as bonding agent, cement, restorative materials, sealants, etc. In some embodiments, the hard tissues that can be treated refer to human and mammalian tissues, including bone, enamel, dentin and cementum. In some embodiments, in particular, the present invented compositions are used to pretreat enamel and dentin. In some embodiments, before the compositions application, the hard tissue is preferably cleaned using traditional methods, i.e., by brushing it with toothpaste, by polishing it with prophy paste, or by abrading it with a bur. In some embodiments, it should be thoroughly rinsed with water and air dried. In some embodiments, during treatment, the invented compositions preferably remained on the treated surface for at least about 5 seconds to about 2 minutes. In some embodiments, more preferably, the pretreatment compositions are allowed to remain on the treated surface for about 10 s to about 30 seconds. In some embodiments, shorter or longer treatment time can also be used to provide the desired results. In some embodiments, after treatment, the compositions should be removed by water rinsing and dried before a dental material application.

### Other Active Agents

In some embodiments, various types of active agents can be included in the composition.

For example, in some embodiments, dental composition of the present disclosure can also contain other active agents to treat the prepared tooth cavity. In some embodiments, when included, the other active agents usually, but not always, include the active agents that are active in the oral cavity against dental diseases, or conditions of the teeth, gums, and the like.

Examples of other active agents that can be employed include fluoride-containing compounds, such as sodium monofluorophosphate, stannous fluoride, zinc fluoride, and combinations thereof.

Examples of other active agents that can be employed include the dyes that can be used to identify dental caries. The dyes for caries identification can include food blue, acid red, alexa fluor 594, amine fluorescein, lucifer yellow CH, and the like.

Examples of addition active agents that can be employed include one or more anti-calculus agents, remineralization agents, stannous sources, antimicrobial agents, antioxidants, antiplaque agents, anti-inflammatory agent, desensitizing agents. Various combinations of such additional active agents may be used if desired. When employed, the other active agents will be used in an amount sufficient to achieve their intended effect. The anti-calculus agents can include phosphate, pyrophosphate, and salts thereof. The remineralization agents can include materials that release calcium ion, phosphorous-containing ions, or both, such as calcium phosphate, hydroxyapatite, calcium chloride, calcium carbonate, and the like. The antimicrobial agents can include a variety of orally acceptable antimicrobial agents, examples include thymol, eugenol, chlorohexidine, silver salts, and the like. When employed, the desensitizing agents can be a variety of orally acceptable desensitizing agents. Examples include potassium citrate, potassium chloride, potassium nitrate, acetyl salicylic acid or salts thereof, salicylic acid or salt thereof, and the like.

### Optional Additives

In some embodiments, dental compositions of the present disclosure include one or more optional additives including surfactants. Various combinations of such additives may be used if desired.

In some embodiments, dental compositions of the present disclosure include a surfactant to stabilize the compositions and improve liquid wetting on the substrate. Oral acceptable surfactants, such as anionic, nonionic, cationic, and amphoteric surfactants, can be used. A combination of surfactants may also be used. Suitable surfactants include but not limited to water-soluble salts of C8-12 alkyl sulfates, sulfonated monoglycerides of C8-20 fatty acids, sarcosinates, taurates and the like. Examples of these includes sodium lauryl sulfate, sodium cocoyl monoglyceride sulfonate, sodium lauroyl sarcosinate, and disodium laureth sulfosuccinate. Suitable nonionic surfactants include without limitation to poloxamers, polyoxyethylene sorbitan esters, fatty alcohol ethxylates, alkylphenol ethoxylates, tertiary amino oxides, tertiary phosphine oxides dialkyl sulfoxides and the like. Suitable amphoteric surfactants include but not limited to derivatives of C8-20 aliphatic secondary and tertiary amines having an anionic group such as carboxylate, sulfate, sulfonate, phosphate or phosphonate. A suitable example is cocamidopropyl betaine. Nonionic surfactants suitable for use in the composition of the present disclosure are generally produced by the condensation of alkylene oxide group with an organic hydrophobic compound which may be aliphatic or alkylaromatic in nature, for example polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixture thereof. In some embodiments, the nonionic surfactant comprises a block copolymer based on ethylene oxide and propylene oxide, such as poloxamer, also known by the trade name Pluronic®. In some embodiments, the nonionic surfactant is poloxamer 407, also known as Pluronic® F127 from BASF.

In some embodiments, the dental composition of the current disclosure can be contained in various types of containers or devices such as a syringe, bottle or vial. In some embodiments, the container or dispenser include but are not limited to 1.2 ml syringes, 3.5 ml syringes, 25 ml syringes or 5 ml bottles.

### NON-LIMITING EMBODIMENTS

1. A dental composition comprising:
a liquid carrier comprising water and at least one organic liquid selected from the group consisting of an alcohol, a polyol, an olefinic monomer, glycerin, propylene glycol, polyethylene glycol, ethanol, acetone;
a thickening agent comprising a polyvinylpyrrolidone having vinylpyrrolidone side groups; and
an active agent,
wherein the dental composition is in the form of a homogeneous gel and is capable of remaining homogeneous for an extended period of time.

2. A dental composition comprising:
a liquid carrier;
a thickening agent to increase viscosity of the dental composition; and
an active agent.

3. A dental composition comprising:
a liquid carrier comprising water and at least one organic liquid;
a thickening system comprising a polyvinylpyrrolidone (PVP); and
an active agent,
wherein the composition forms a homogeneous gel that remains substantially homogeneous under storage conditions.

4. The dental composition of embodiment 2, wherein the thickening agent comprises a polyvinylpyrrolidone.

5. The dental composition of embodiment 4, the polyvinylpyrrolidone has a vinylpyrrolidone side group.

6. The dental composition of any one of embodiments 4-5, the polyvinylpyrrolidone has a plurality of vinylpyrrolidone side groups.

7. The dental composition of any one of embodiments 2, wherein the liquid carrier comprises at least one organic liquid selected from the group consisting of water, an alcohol, a polyol, an olefinic monomer, glycerin, propylene glycol, polyethylene glycol, ethanol, acetone.

8. The dental composition of any one of embodiments 2-6, wherein the dental composition is in the form of a homogeneous gel.

9. The dental composition of any one of embodiments 2-8, wherein the dental composition is capable of remaining homogeneous for a period of time.

10. The dental composition of any one of embodiments 1-9, wherein the polyvinylpyrrolidone comprises a homopolymer or a copolymer of one or more monomers having one or more vinylpyrrolidone groups.

11. The dental composition of any one of embodiments 1-10, wherein the composition maintains homogeneity and viscosity stability under at least one of:
(i) a pH less than about 2,
(ii) a pH greater than about 10, or
(iii) a solution containing greater than about 20 wt % dissolved salts.

12. The dental composition of any one of embodiments 1-11, wherein the composition has a viscosity sufficient to increase residence time of the composition on a tooth surface and reduce uncontrolled spreading relative to an otherwise identical composition lacking a thickening agent.

13. The composition of any one of embodiments 1, wherein the composition comprises a polyvinylpyrrolidone (PVP) homopolymer.

14. The composition of any one of embodiments 1-11, wherein the composition has a viscosity from about 50 cP to about 10,000 cP.

15. The composition of embodiment 14, wherein the viscosity is from about 100 cP to about 5,000 cP.

16. The composition of embodiment 14, wherein the viscosity is from about 300 cP to about 1,500 cP.

17. The composition of any one of embodiments 1-11, wherein the composition exhibits shear-thinning behavior.

18. The composition of any one of embodiments 1-11, wherein the composition exhibits thixotropic behavior.

19. The composition of any one of embodiments 1-11, wherein the composition maintains homogeneity after storage at a temperature from about 42° C. to about 60° C.

20. The composition of any one of embodiments 1-11, wherein the composition maintains at least about 80% of its initial viscosity after storage at about 60° C. for at least 4 weeks.

21. The composition of any one of embodiments 1-11, wherein the composition exhibits reduced syneresis relative to a silica-thickened composition.

22. The composition of any one of embodiments 1-11, wherein the composition remains substantially homogeneous at a pH less than about 2.

23. The composition of any one of embodiments 1-11, wherein the composition remains substantially homogeneous at a pH greater than about 10.

24. The composition of any one of embodiments 1-11, wherein the composition remains substantially homogeneous in the presence of greater than about 20 wt % dissolved salts.

25. The composition of any one of embodiments 1-11, wherein the active agent comprises an acid.

26. The composition of embodiment 25, wherein the acid comprises phosphoric acid.

27. The composition of embodiment 25, wherein the acid is present in an amount from about 20 wt % to about 60 wt % of the composition.

28. The composition of embodiment 27, wherein the acid is present in an amount from about 30 wt % to about 50 wt % of the composition.

29. The composition of any one of embodiments 1-11, wherein the active agent comprises an aldehyde.

30. The composition of embodiment 29, wherein the aldehyde comprises glutaraldehyde.

31. The composition of embodiment 29, wherein the aldehyde is present in an amount from about 3 wt % to about 15 wt % of the composition.

32. The composition of any one of embodiments 1-11, wherein the active agent comprises silver diamine fluoride.

33. The composition of embodiment 32, wherein the silver diamine fluoride is present in an amount from about 1 wt % to about 50 wt % of the composition.

34. The composition of any one of embodiments 1-11, wherein the active agent comprises an antimicrobial agent, an anti-inflammatory agent, a desensitizing agent, or a remineralization agent.

35. The composition of embodiment 34, wherein the antimicrobial agent comprises chlorhexidine.

36. The composition of any one of embodiments 1-11, wherein the liquid carrier comprises water and at least one organic liquid selected from glycerin, propylene glycol, polyethylene glycol, ethanol, or acetone.

37. The composition of embodiment 36, wherein the liquid carrier is present in an amount from about 20 wt % to about 60 wt % of the composition.

38. The composition of any one of embodiments 1-11, wherein the composition comprises a polyol selected from glycerin, propylene glycol, or polyethylene glycol.

39. The composition of any one of embodiments 1-11, further comprising an olefinic monomer.

40. The composition of embodiment 39, wherein the olefinic monomer comprises HEMA.

41. The composition of embodiment 40, wherein the HEMA is present in an amount from about 25 wt % to about 45 wt % of the composition.

42. The composition of any one of embodiments 1-11, wherein the composition exhibits improved localization on a tooth surface compared to a composition lacking the thickening system.

43. The composition of any one of embodiments 1-11, wherein the composition reduces spread to adjacent soft tissue relative to a lower-viscosity composition.

44. The composition of any one of embodiments 1-11, wherein the composition provides controlled residence time on a treated surface.

45. The composition of any one of embodiments 1-11, wherein the composition is configured for use as a dental etchant.

46. The composition of any one of embodiments 1-11, wherein the composition is configured for use as a dental disinfectant.

47. The composition of any one of embodiments 1-11, wherein the composition is configured for pretreatment of a tooth surface prior to restoration.

48. The composition of any one of embodiments 1-11, wherein the composition is in the form of a gel.

49. The dental composition of any one of embodiments 1-48, wherein the liquid carrier is present in a range of about 20% to about 60% with respect to weight of the dental composition.

50. The composition of any one of embodiments 1-49, further comprising at least organic liquid having a concentration in a range of about 0.1% to about 50% with respect to weight of the dental composition.

51. The composition of any one of embodiments 1-50, wherein the the active agent comprises an aldehyde, an olefinic monomer, or a combination of the aldehyde and the olefinic monomer.

52. The composition of any one of embodiments 51, wherein the aldehyde is selected from formaldehyde, propionaldehyde, butyraldehyde, glutaraldehyde, glycolaldehyde, benzaldehyde, or a combination thereof.

53. The composition of any one of embodiments 51-52, wherein the olefinic monomer comprises an ester of acrylic or methacrylic acid.

54. The composition of any one of embodiments 1-53, wherein the active agent comprises silver diamine fluoride.

55. The composition of any one of embodiments 1-54, wherein the active agent comprises silver diamine fluoride in a range of from about 1% to about 65% with respect to weight of the dental composition.

56. The dental composition of any one of embodiments 1-54, wherein the active agent comprises silver diamine fluoride in a range of from about 1% to about 60% with respect to weight of the dental composition.

57. The dental composition of any one of embodiments 1-54, wherein the active agent comprises silver diamine fluoride in a range of from about 1% to about 55% with respect to weight of the dental composition.

58. The dental composition of any one of embodiments 1-54, wherein the active agent comprises silver diamine fluoride in a range of from about 1% to about 50% with respect to weight of the dental composition.

59. The dental composition as defined in embodiment 1-58, wherein the dental composition exhibits relatively enhanced handleability and storage stability compared to the dental composition without the thickening agent.

60. The dental composition of any one of embodiments 1-59, wherein the thickening agent is present in an amount ranging from about 0.1 wt % to about 30 wt % of the composition.

61. The dental composition of any one of embodiments 1-59, wherein the thickening agent is present in an amount ranging from about 0.1 wt % to about 20 wt % of the composition.

62. The dental composition of any one of embodiments 1-59, wherein the thickening agent is present in an amount ranging from about 0.1 wt % to about 15 wt % of the composition.

63. The dental composition of any one of embodiments 1-59, wherein the thickening agent is present in an amount ranging from about 0.5 wt % to about 30 wt % of the composition.

64. The dental composition of any one of embodiments 1-59, wherein the thickening agent is present in an amount ranging from about 0.5 wt % to about 20 wt % of the composition.

65. The dental composition of any one of embodiments 1-59, wherein the thickening agent is present in an amount ranging from about 0.5 wt % to about 15 wt % of the composition.

66. The dental composition of any one of embodiments 1-65, wherein the dental composition exhibits viscosity of from about 50 cP to about 10,000 cP.

67. The dental composition of any one of embodiments 1-62, wherein the dental composition exhibits viscosity of from about 50 cP to about 9,000 cP.

68. The dental composition of any one of embodiments 1-62, wherein the dental composition exhibits viscosity of from about 50 cP to about 8,000 cP.

69. The dental composition of any one of embodiments 1-62, wherein the dental composition exhibits viscosity of from about 50 cP to about 7,000 cP.

70. The dental composition of any one of embodiments 1-62, wherein the dental composition exhibits viscosity of from about 50 cP to about 5,000 cP.

71. The dental composition of any one of embodiments 1-62, wherein the dental composition exhibits viscosity of from about 50 cP to about 4,000 cP.

72. The dental composition of any one of embodiments 1-62, wherein the dental composition exhibits viscosity of from about 50 cP to about 3,000 cP.

73. The dental composition of any one of embodiments 1-62, wherein the dental composition exhibits viscosity of from about 50 cP to about 2,000 cP.

74. The dental composition of any one of embodiments 1-73, wherein the active agent comprises an anti-calculus agent, a remineralization agent, an antimicrobial agent, an antioxidant, an antiplaque agent, an anti-inflammatory agent, a desensitizing agent, and a combination thereof.

75. The dental composition of any one of embodiments 1-74, the dental composition is to be used as a pretreatment of a surface of tooth in dental restoration.

76. The dental composition of any one of embodiments 1-74, further comprising HEMA.

77. The dental composition of any one of embodiments 76, the amount of HEMA in the dental composition is from about 25 wt. % to about 45 wt. % with respect to the weight of the dental composition.

78. The dental composition of any one of embodiments 76-77, the amount of the polyvinylpyrrolidone present in the dental composition is from about 1 wt. % to about 12 wt. % with respect to the weight of the dental composition.

79. The dental composition of any one of embodiments 76-77, the amount of the polyvinylpyrrolidone present in the dental composition is from about 1 wt. % to about 8 wt. % with respect to the weight of the dental composition.

80. The dental composition of any one of embodiments 76-77, the amount of the polyvinylpyrrolidone present in the dental composition is from about 2 wt. % to about 12 wt. % with respect to the weight of the dental composition.

81. The dental composition of any one of embodiments 76-77, the amount of the polyvinylpyrrolidone present in the dental composition is from about 2 wt. % to about 10 wt. % with respect to the weight of the dental composition.

82. The dental composition of any one of embodiments 76-77, the amount of the polyvinylpyrrolidone present in the dental composition is from about 2 wt. % to about 8 wt. % with respect to weight of the dental composition.

83. The dental composition of any one of embodiments 76-82, wherein the dental composition exhibits adhesion strength of the dental composition to dentin of at least about 25 MPa.

84. The dental composition of any one of embodiments 76-82, wherein the dental composition exhibits adhesion strength of the dental composition to dentin of at least about 26 MPa.

85. The dental composition of any one of embodiments 76-82, wherein the dental composition exhibits adhesion strength of the dental composition to dentin of at least about 27 MPa.

86. The dental composition of any one of embodiments 76-82, wherein the dental composition exhibits adhesion strength of the dental composition to dentin of at least about 28 MPa.

87. The dental composition of any one of embodiments 76-82, wherein the dental composition exhibits adhesion strength of the dental composition to dentin of at least about 29 MPa.

88. The dental composition of any one of embodiments 76-82, wherein the dental composition exhibits adhesion strength of the dental composition to dentin of at least about 29.5 MPa.

89. The dental composition of any one of embodiments 76-82, wherein the dental composition exhibits adhesion strength of the dental composition to dentin of at least about 30 MPa.

90. The dental composition of any one of embodiments 76-82, wherein the dental composition exhibits adhesion strength of the dental composition to dentin of at least about 30.5 MPa.

91. The dental composition of any one of embodiments 76-82, wherein the dental composition exhibits adhesion strength of the dental composition to dentin of at least about 31 MPa.

92. The dental composition of any one of embodiments 76-82, wherein the dental composition exhibits adhesion strength of the dental composition to dentin of at least about 31.5 MPa.

93. The dental composition of any one of embodiments 76-82, wherein the dental composition exhibits adhesion strength of the dental composition to dentin of at least about 32 MPa.

94. The dental composition of any one of embodiments 76-82, wherein the dental composition exhibits adhesion strength of the dental composition to dentin of at least about 32.5 MPa.

95. The dental composition of any one of embodiments 76-82, wherein the dental composition exhibits adhesion strength of the dental composition to dentin of at least about 33 MPa.

96. The dental composition of any one of embodiments 76-82, wherein the dental composition exhibits adhesion strength of the dental composition to dentin of at least about 33.5 MPa.

97. The dental composition of any one of embodiments 76-82, wherein the dental composition exhibits adhesion strength of the dental composition to dentin of at least about 34 MPa.

98. The dental composition of any one of embodiments 76-82, wherein the dental composition exhibits adhesion strength of the dental composition to dentin of at least about 35 MPa.

99. The dental composition of any one of embodiments 76-82, wherein the dental composition exhibits adhesion strength of the dental composition to dentin of at least about 36 MPa.

100. The dental composition of any one of embodiments 76-82, wherein the dental composition exhibits adhesion strength of the dental composition to dentin of at least about 37 MPa.

101. The dental composition of any one of embodiments 76-82, wherein the dental composition exhibits adhesion strength of the dental composition to dentin of at least about 38 MPa.

102. The dental composition of any one of embodiments 76-82, wherein the dental composition exhibits adhesion strength of the dental composition to dentin of at least about 39 MPa.

103. The dental composition of any one of embodiments 76-82, wherein the dental composition exhibits adhesion strength of the dental composition to dentin of at least about 41 MPa.

104. The dental composition of any one of embodiments 76-82, wherein the dental composition exhibits adhesion strength of the dental composition to dentin of at least about 42 MPa.

105. The dental composition of any one of embodiments 76-82, wherein the dental composition exhibits adhesion strength of the dental composition to dentin of at least about 43 MPa.

106. The dental composition of any one of embodiments 76-82, wherein the dental composition exhibits adhesion strength of the dental composition to dentin of at least about 44 MPa.

107. The dental composition of any one of embodiments 76-82, wherein the dental composition exhibits adhesion strength of the dental composition to dentin of at least about 45 MPa.

108. The dental composition of any one of embodiments 76-82, wherein the dental composition exhibits adhesion strength of the dental composition to dentin of from about 25 to about 100 MPa.

109. The dental composition of any one of embodiments 76-82, wherein the dental composition exhibits adhesion strength of the dental composition to dentin of from about 26 to about 100 MPa.

110. The dental composition of any one of embodiments 76-82, wherein the dental composition exhibits adhesion strength of the dental composition to dentin of from about 28 to about 100 MPa.

111. The dental composition of any one of embodiments 76-82, wherein the dental composition exhibits adhesion strength of the dental composition to dentin of from about 30 to about 100 MPa.

112. The dental composition of any one of embodiments 76-82, wherein the dental composition exhibits adhesion strength of the dental composition to dentin of from about 25 to about 70 MPa.

113. The dental composition of any one of embodiments 76-82, wherein the dental composition exhibits adhesion strength of the dental composition to dentin of from about 26 to about 70 MPa.

114. The dental composition of any one of embodiments 76-82, wherein the dental composition exhibits adhesion strength of the dental composition to dentin of from about 28 to about 70 MPa.

115. The dental composition of any one of embodiments 76-82, wherein the dental composition exhibits adhesion strength of the dental composition to dentin of from about 30 to about 70 MPa.

116. The dental composition of any one of embodiments 76-82, wherein the dental composition exhibits adhesion strength of the dental composition to dentin of from about 25 to about 50 MPa.

117. The dental composition of any one of embodiments 76-82, wherein the dental composition exhibits adhesion strength of the dental composition to dentin of from about 26 to about 50 MPa.

118. The dental composition of any one of embodiments 76-82, wherein the dental composition exhibits adhesion strength of the dental composition to dentin of from about 28 to about 50 MPa.

119. The dental composition of any one of embodiments 76-82, wherein the dental composition exhibits adhesion strength of the dental composition to dentin of from about 30 to about 50 MPa.

120. The dental composition of any one of embodiments 76-82, wherein the dental composition substantially maintains viscosity or non-paste form after the dental composition is stored from about 42° C. to about 60° C. for a period of time.

121. The dental composition of any one of embodiments 1-10, wherein the active agent comprises a mineral acid selected from phosphoric acid, nitric acid, hydrochloric acid or sulfuric acid.

122. The dental composition of any one of embodiments 121, wherein the active agent comprises phosphoric acid.

123. The dental composition of any one of embodiments 121-122, wherein the amount of the phosphoric acid in the dental composition is in a range of about 20% to about 80% with respect to weight of the dental composition.

124. The dental composition of any one of embodiments 121-122, wherein the amount of the phosphoric acid in the dental composition is 85% phosphoric acid in a range of about 20% to about 80% with respect to weight of the dental composition.

125. The dental composition of any one of embodiments 121-122, wherein the amount of the phosphoric acid in the dental composition is in a range of about 20% to about 70% with respect to weight of the dental composition.

126. The dental composition of any one of embodiments 121-122, wherein the amount of the phosphoric acid in the dental composition is 85% phosphoric acid in a range of about 20% to about 70% with respect to weight of the dental composition.

127. The dental composition of any one of embodiments 121-122, wherein the amount of the phosphoric acid in the dental composition is in a range of about 20% to about 65% with respect to weight of the dental composition.

128. The dental composition of any one of embodiments 121-122, wherein the amount of the phosphoric acid in the dental composition is 85% phosphoric acid in a range of about 20% to about 65% with respect to weight of the dental composition.

129. The dental composition of any one of embodiments 121-122, wherein the amount of the phosphoric acid in the dental composition is in a range of about 20% to about 50% with respect to weight of the dental composition.

130. The dental composition of any one of embodiments 121-122, wherein the amount of the phosphoric acid in the dental composition is 85% phosphoric acid in a range of about 20% to about 50% with respect to weight of the dental composition.

131. The dental composition of any one of embodiments 121-122, wherein the amount of the phosphoric acid in the dental composition is in a range of about 35% to about 50% with respect to weight of the dental composition.

132. The dental composition of any one of embodiments 121-122, wherein the amount of the phosphoric acid in the dental composition is 85% phosphoric acid in a range of about 35% to about 50% with respect to weight of the dental composition.

133. The dental composition of any one of embodiments 121-122, wherein the amount of the phosphoric acid in the dental composition is in a range of about 35% to about 45% with respect to weight of the dental composition.

134. The dental composition of any one of embodiments 121-122, wherein the amount of the phosphoric acid in the dental composition is 85% phosphoric acid in a range of about 35% to about 45% with respect to weight of the dental composition.

135. The dental composition of any one of embodiments 121-134, the amount of the polyvinylpyrrolidone present in the dental composition is from about 1 wt. % to about 10 wt. % with respect to the weight of the dental composition.

136. The dental composition of any one of embodiments 121-134, the amount of the polyvinylpyrrolidone present in the dental composition is from about 1 wt. % to about 8 wt. % with respect to the weight of the dental composition.

137. The dental composition of any one of embodiments 121-134, the amount of the polyvinylpyrrolidone present in the dental composition is from about 1 wt. % to about 7 wt. % with respect to the weight of the dental composition.

138. The dental composition of any one of embodiments 121-134, the amount of the polyvinylpyrrolidone present in the dental composition is from about 2 wt. % to about 10 wt. % with respect to the weight of the dental composition.

139. The dental composition of any one of embodiments 121-134, the amount of the polyvinylpyrrolidone present in the dental composition is from about 2 wt. % to about 8 wt. % with respect to the weight of the dental composition.

140. The dental composition of any one of embodiments 121-134, the amount of the polyvinylpyrrolidone present in the dental composition is from about 2 wt. % to about 7 wt. % with respect to weight of the dental composition.

141. The dental composition of any one of embodiments 121-140, the dental composition exhibits the viscosity of from about 50 cP to about 1500 cP.

142. The dental composition of any one of embodiments 121-141, the dental composition exhibits the viscosity of from about 50 cP to about 1400 cP.

143. The dental composition of any one of embodiments 121-141, the dental composition exhibits the viscosity of from about 50 cP to about 1300 cP.

144. The dental composition of any one of embodiments 121-141, the dental composition exhibits the viscosity of from about 50 cP to about 300 cP.

145. The dental composition of any one of embodiments 121-141, the dental composition exhibits the viscosity of from about 300 cP to about 800 cP.

146. The dental composition of any one of embodiments 121-141, the dental composition exhibits the viscosity of from about 350 cP to about 700 cP.

147. The dental composition of any one of embodiments 121-141, the dental composition exhibits the viscosity of from about 500 cP to about 1500 cP.

148. The dental composition of any one of embodiments 121-141, the dental composition exhibits the viscosity of from about 600 cP to about 1400 cP.

149. The dental composition of any one of embodiments 121-141, the dental composition exhibits the viscosity of from about 700 cP to about 1400 cP.

150. The dental composition of any one of embodiments 121-140, the dental composition exhibits the viscosity of from about 100 cP to about 1500 after the dental composition is stored at around 60° C. for about 8 weeks.

151. The dental composition of any one of embodiments 121-141, the dental composition exhibits the viscosity of from about 100 cP to about 1400 after the dental composition is stored at around 60° C. for about 8 weeks.

152. The dental composition of any one of embodiments 121-141, the dental composition exhibits the viscosity of from about 100 cP to about 1300 after the dental composition is stored at around 60° C. for about 8 weeks.

153. The dental composition of any one of embodiments 121-141, the dental composition exhibits the viscosity of from about 100 cP to about 300 after the dental composition is stored at around 60° C. for about 8 weeks.

154. The dental composition of any one of embodiments 121-141, the dental composition exhibits the viscosity of from about 350 cP to about 800 after the dental composition is stored at around 60° C. for about 8 weeks.

155. The dental composition of any one of embodiments 121-141, the dental composition exhibits the viscosity of from about 400 cP to about 700 after the dental composition is stored at around 60° C. for about 8 weeks.

156. The dental composition of any one of embodiments 121-141, the dental composition exhibits the viscosity of from about 550 cP to about 1500 after the dental composition is stored at around 60° C. for about 8 weeks.

157. The dental composition of any one of embodiments 121-141, the dental composition exhibits the viscosity of from about 650 cP to about 1400 after the dental composition is stored at around 60° C. for about 8 weeks.

158. The dental composition of any one of embodiments 121-141, the dental composition exhibits the viscosity of from about 800 cP to about 1400 after the dental composition is stored at around 60° C. for about 8 weeks.

159. The dental composition of any one of embodiments 121-158, the viscosity of the dental composition does not increase or increases less than about 1000 cP after the dental composition is stored at around 60° C. for about 8 weeks.

160. The dental composition of any one of embodiments 121-158, the viscosity of the dental composition does not increase or increases less than about 700 cP after the dental composition is stored at around 60° C. for about 8 weeks.

161. The dental composition of any one of embodiments 121-158, the viscosity of the dental composition does not increase or increases less than about 500 cP after the dental composition is stored at around 60° C. for about 8 weeks.

162. The dental composition of any one of embodiments 121-158, the viscosity of the dental composition does not increase or increases less than about 400 cP after the dental composition is stored at around 60° C. for about 8 weeks.

163. The dental composition of any one of embodiments 121-162, the pH of the dental composition is substantially maintained, or decreases, or does not increase after the dental composition is stored at around 60° C. for about 4 weeks.

164. The dental composition of any one of embodiments 121-162, the pH of the dental composition decreases by about 0.05 or more and less than about 2 after the dental composition is stored at around 60° C. for about 4 weeks.

165. The dental composition of any one of embodiments 121-162, the pH of the dental composition decreases by about 0.1 or more and less than about 2 after the dental composition is stored at around 60° C. for about 4 weeks.

166. The dental composition of any one of embodiments 121-162, the pH of the dental composition decreases by about 0.05 or more and less than about 1 after the dental composition is stored at around 60° C. for about 4 weeks.

167. The dental composition of any one of embodiments 121-162, the pH of the dental composition decreases by about 0.1 or more and less than about 1 after the dental composition is stored at around 60° C. for about 4 weeks.

168. The dental composition of any one of embodiments 121-162, the pH of the dental composition decreases by about 0.05 or more and less than about 0.5 after the dental composition is stored at around 60° C. for about 4 weeks.

169. The dental composition of any one of embodiments 121-162, the pH of the dental composition decreases by about 0.1 or more and less than about 0.5 after the dental composition is stored at around 60° C. for about 4 weeks.

170. A method comprising applying dental composition of any one of embodiments 1-169 to a tooth surface.

171. A device to apply the dental composition of any one of embodiments 1-170 to a tooth surface, comprising:
a container configured to contain the dental composition.

### Experimental Examples

The following examples further describe and demonstrate the preferred embodiments within the scope of the present disclosure. The examples are given solely for the purpose of illustration and are not to be construed as limitations of the present disclosure since many variations thereof as possible without departing from its spirit and scope. The dental compositions directed to the present disclosure can be prepared by conventional methods known to persons of ordinary skill in the art, such as, mixing or blending using a speedmixer or homogenizer.

These and other aspects of the present disclosure are described by the following examples which should not be treated as limiting in scope. Unless otherwise indicated, all ratios, parts and percentages are by weight.

### Example Composition Preparations

Compositions were prepared as follows.

Various compositions were formulated and mixed or blended using a speedmixer or homogenizer. Various compositions included at least one of the listed agents in Table 1, in various amounts, for example, amounts in the weight percent (wt. %) ranges, with respect to the weight of the composition, including the example compositions Example 1. For example, Example 1 and Comparative Examples 2 and 3 were prepared as indicated in Table 2. Examples 4-6 and Comparative Examples 7 and 8 were prepared as indicated in Table 4. Examples 4 through 6 were the dental etching compositions directed to the present disclosure thickened by Flexithix PVP. Comparative Example 7 was thickened by hydroxyethyl cellulose (Natrosol 250 HEC). Comparative Example 8 was thickened by cellulose nanocrystals.

TABLE 1
List of compounds for example compositions.

| Description | Source | Location |
|---|---|---|
| PVP, Flexithix | Ashland | Wilmington, DE |
| Natrosol 250H | Ashland | Wilmington, DE |
| hydroxyethyl cellulose | | |
| Cellulose nanocrystals | CelluForce | Montreal, Canada |
| Aerosil 200 | Evonik | Piscataway, NJ |
| HDK-N20P | Wacker | Charleston, TN |
| Hydroxyethyl | Gelest | Gen Rock, PA |
| methacrylate | | |
| Glutaric dialdehyde | Spectrum Chemical | New Brunswick, NJ |
| Phosphoric acid | Spectrum Chemical | New Brunswick, NJ |
| Glycerin | Wilmar International | Wilmar Stockton, CA |
| Propylene glycol | Greenfield Global | Berkeley, CA |
| Acid Red | Pylam Products | Tempe, AZ |

TABLE 2
Example Compositions.

| | | Comparative | Comparative |
|---|---|---|---|
| | Example 1 | Example 2 | Example 3 |
| Component | (wt. %) | (wt. %) | (wt. %) |
| Flexithix PVP | 7 | 0 | 0 |
| Fumed silica | 0 | 0 | 8 |
| (Aerosil 200) | | | |
| Fumed silica | 0 | 10 | 0 |
| (HDK-N20P) | | | |
| PEG 600 | 0 | 1 | 1 |
| Propylene glycol | 0.124 | 0 | 0 |
| Glutaraldehyde | 5 | 5 | 5 |
| HEMA | 36 | 36 | 36 |
| Dye | 0.001 | 0.001 | 0.001 |
| Water | QS to 100 | QS to 100 | QS to 100 |

### Properties and Characteristics of Compositions Including HEMA

Different properties and characteristics of Example 1, Comparative Examples 2 and 3, and GLUMA® POWERGEL were tested. The results of testings were indicated in Table 3.

The physical appearances of Example 1, Comparative Examples 2 and 3, and GLUMA® POWERGEL were evaluated and compared. Example 1 exhibited a relatively more homogeneous gel while Comparative Examples 2 and 3, and GLUMA® POWERGEL exhibited thick paste.

Stability testing of the dental composition of present disclosure was conducted at 42° C. or 60° C. to determine if viscosity or pH was changed over time. The dental compositions were filled into 3 cc clear HDPE Luer-lock syringes affixed with plungers. The syringes were placed into an oven at 42 or 60° C. for aging. For the dental composition of the current disclosure, Example 1 above, exhibited a non-paste form without paste thickness and without paste-like property and substantially better flowability than and could be dispensed relatively in an easy and controllable mode and was storage stable, compared to Comparative Examples 2 and 3 and GLUMA® PowerGel which exhibited thick paste-like property and poor flowability.

Shear bond strength on human dentin treated with Example 1 was compared to non-treated and GLUMA® PowerGel treated specimens. Before treatment, the human dentin specimens were polished with 320-grit SiC papers, rinsed thoroughly with water and air dried briefly. An etchant solution of 37% phosphoric acid (SURE ETCH™ liquid, Zest Dental Solutions) was applied on dentin surface for 15 seconds and then rinsed off. The Example 1 and GLUMA® PowerGel was applied on the etched dentin surface for 30 s respectively and then thoroughly rinsed off After treatment, a layer of self-adhesive, dental adhesive (PRELUDE ONE™, Zest Dental Solution) was applied and light cured for 10 seconds. The dentin specimen was then held securely by a bonding jig (Ultradent Inc., UT) with a cylindrical mold (Φ=2.38 mm). A dual-cure flowable resin-cement (CEMEZ™, Zest Dental Solutions) was condensed inside the mold and light-cured for 20 seconds using a dental curing light (Ivocla vivadent blue phase, Intensity=1585 mW/cm²). All specimens were then conditioned at 37° C. for 24 hours. Their shear bond strength was tested on an Instron mechanical tester (Model 4411, Instron, MA) using a notched (semi-circular) edge at a crosshead speed of 1.0 mm/min. The test results is indicated in Table 3. Without dentin treatment, the shear bond strength of self-adhesive, dental adhesive on dentin was 31.0±5.9 MPa. With the treatment of Example 1, the shear bond strength was 31.5±5.9 MPa which is unexpectedly significantly higher than the treatment of GLUMA® PowerGel.

TABLE 3

| | Physical appearance | | Dispensed by 22 g needle | | Adhesion |
|---|---|---|---|---|---|
| | | 22 wk | | 22 wk | to Dentin |
| Description | 0 d | @42° C. | 0 d | @42° C. | (MPa) |

| Example 1 | Homogeneous gel | Easy and controllable | 31.5 ± 5.9 |
|---|---|---|---|
| Ex. 2 | Thick paste | Hard | NT* |
| Ex. 3 | Thick paste | Hard | NT |
| GLUMA ® | Thick paste | Hard | 26.7 ± 2.2 |
| PowerGel | | | |

*NT = Not Tested

### Example 3: Properties and Characteristics of Acidic Compositions

pH testing was carried out on the example compositions using a standard pH meter after calibration. The pH meter used was ACCUMET model AB250 from Fisher Scientific. The measurement was performed by inserting a pH probe into the composition, waiting until the reading is stable and then recording the pH value. The pH values were listed in the example Table 5.

The pH of Examples 4 through 6 and Comparative Examples 7 and 8 was between 0 and 1 (Table 5). The resulting etching compositions of Examples 4 through 6 were found to be stable and substantially maintained viscosity and pH for over 8 weeks at 60° C., such that the pH of etching compositions was still between 0 and 1. They did not show syneresis after 8 weeks of aging at 60° C. The etching compositions of Examples 4 through 6 were relatively easily dispensed from dental 25-gauge needle tips. In comparison, the hydroxyethyl cellulose thickened composition (Comparative Example 7) was relatively substantially degraded with lower viscosity and waterier after aging. Both Comparative Example 8 thickened by cellulose nanocrystal and ULTRA-ETCH™ etchant thickened by fumed silica showed syneresis, i.e., partial liquid was extracted from the gel.

TABLE 4

| | | | | Compar- | Compar- |
|---|---|---|---|---|---|
| | | | | ative | ative |
| | Exam- | Exam- | Exam- | Exam- | Exam- |
| | ple 4 | ple 5 | ple 6 | ple 7 | ple 8 |
| Component | (wt. %) | (wt. %) | (wt. %) | (wt. %) | (wt. %) |
| Flexithix | 3 | 4 | 5 | 0 | 0 |
| PVP | | | | | |
| Natrosol | 0 | 0 | 0 | 1.7 | 0 |
| 250HEC | | | | | |
| Cellulose | 0 | 0 | 0 | 0 | 6 |
| nanocrystal | | | | | |
| glycerin | 4.8 | 4.8 | 4.8 | 4.8 | 4.8 |
| Propylene | 0.4 | 0.4 | 0.4 | 0.4 | 0.4 |
| glycol | | | | | |
| Phosphoric | 41.9 | 41.9 | 41.9 | 41.9 | 41.9 |
| acid (85%) | | | | | |
| Acid Red | 0.004 | 0.004 | 0.004 | 0.004 | 0.004 |
| Water | QS to 100 | QS to 100 | QS to 100 | QS to 100 | QS to 100 |

TABLE 5

| | pH | | Viscosity (cP.) | | Syneresis (Y/N) | |
|---|---|---|---|---|---|---|
| Description | 0 d | 4 wk@60° C. | 0 d | 8 wk@60° C. | 0 d | 8 wk@60° C. |
| Example 4 | 0.53 | 0.11 | 61 | 143 | N | N |
| Example 5 | 0.39 | 0.27 | 413 | 620 | N | N |
| Example 6 | 0.53 | 0.21 | 880 | 1248 | N | N |
| Comparative | 0.35 | NA | 1753 | NA | N | NA |
| Example 7 | | | | | | |
| Comparative | 0.36 | NA | 3040 | NA | N | Y |
| Example 8 | | | | | | |
| ULTRA-ETCH ™ | 0.25 | 0.34 | 96 | 139 | N | Y |

The shear bond strength test of SUREETCH™ liquid etchant, Examples 4 through 6 and ULTRA-ETCH™ etchant was conducted as follows: bovine enamel and human dentin specimens (N=7 for each group) were embedded in cold-cured acrylics. The enamel and dentin surfaces were polished with 320-grit SiC papers, rinsed thoroughly with water and air dried briefly. The etchant gel or liquid was applied on enamel or dentin surface for 15 s and then rinsed off. The enamel or dentin surface was then held securely by a bonding jig (Ultradent Inc., UT) with a cylindrical mold (Φ=2.38 mm). An experimental self-adhesive composite was condensed inside the mold for 10 s and then light-cured for 20 s using a dental curing light (Ivocla vivadent blue phase, Intensity=1585 mW/cm²). All specimens were then conditioned at 37° C. for 24 hours. Their shear bond strength was tested on the Instron mechanical tester as mentioned above.

The shear bond strength of etched enamel was similar (Table 6). On dentin, etching with the Ex. 4-6 and ULTRA-ETCH™ showed significant higher bond strength than etching with SUREETCH™ liquid.

TABLE 6

| | SureEtch ™ | | Example | Example | | Example | | Ultra- | |
|---|---|---|---|---|---|---|---|---|---|
| Shear bond strength (MPa) | Liquid | | 4 | 5 | | 6 | | Etch ™ | |
| Adhesion to Enamel (s.d.) | 28.8 | (4.1) | 29.7 (6.3) | 28.4 | (6.7) | 26.4 | (6.8) | 27.9 | (8.5) |
| Adhesion to Dentin (s.d.) | 3.3 | (2.7) | 12.2 (4.6) | 7.3 | (2.6) | 9.3 | (4.0) | 6.8 | (2.2) |

Of note, example embodiments of the disclosure described above do not limit the scope of the present disclosure since these embodiments are merely examples of the embodiments of the present disclosure. Any equivalent embodiments are intended to be within the scope of this present disclosure. Indeed, various modifications of the disclosure, in addition to those shown and described herein, such as alternative useful combinations of the elements described, may become apparent to those skilled in the art from the description. Such modifications and embodiments are also intended to fall within the scope of the appended claims.

What is claimed is:

1. A dental composition comprising:
a liquid carrier comprising water and at least one organic liquid selected from the group consisting of an alcohol, a polyol, an olefinic monomer, glycerin, propylene glycol, polyethylene glycol, ethanol, acetone;
a thickening agent comprising a polyvinylpyrrolidone having vinylpyrrolidone side groups; and
an active agent,
wherein the composition has a viscosity sufficient to increase residence time of the composition on a tooth surface and reduce uncontrolled spreading relative to an otherwise identical composition lacking a thickening agent,
wherein the dental composition is in the form of a homogeneous gel and is capable of remaining homogeneous for at least about 8 weeks,
wherein the dental composition exhibits relatively more storage stability compared to the dental composition without the thickening agent.

2. The dental composition of claim 1, the polyvinylpyrrolidone has a plurality of vinylpyrrolidone side groups.

3. The dental composition of claim 1, wherein the polyvinylpyrrolidone comprises a homopolymer or a copolymer of one or more monomers having one or more vinylpyrrolidone groups.

4. The dental composition of claim 1, wherein the composition maintains homogeneity and viscosity stability under at least one of:
(i) a pH less than about 2,
(ii) a pH greater than about 10, or
(iii) a solution containing greater than about 20 wt % dissolved salts.

5. The dental composition of claim 1, wherein the thickening agent is present in an amount ranging from about 0.1 wt % to about 30 wt % of the composition.

6. The dental composition of claim 1, wherein the active agent comprises an anti-calculus agent, a remineralization agent, an antimicrobial agent, an antioxidant, an antiplaque agent, an anti-inflammatory agent, a desensitizing agent, and a combination thereof.

7. The dental composition of claim 1, further comprising HEMA.

8. The dental composition of claim 7, the amount of HEMA in the dental composition is from about 25 wt. % to about 45 wt. % with respect to the weight of the dental composition.

9. The dental composition of claim 7, the amount of the polyvinylpyrrolidone present in the dental composition is from about 1 wt. % to about 12 wt. % with respect to the weight of the dental composition.

10. The dental composition of claim 1, wherein the dental composition exhibits adhesion strength of the dental composition to dentin of at least about 25 MPa.

11. The dental composition of claim 1, wherein the dental composition exhibits adhesion strength of the dental composition to dentin of from about 25 to about 100 MPa.

12. The dental composition of claim 1, wherein the dental composition substantially maintains viscosity or non-paste form after the dental composition is stored from about 42° C. to about 60° C. for a period of time.

13. The dental composition of claim 1, wherein the active agent comprises phosphoric acid.

14. The dental composition of claim 1, wherein the amount of the phosphoric acid in the dental composition is in a range of about 20% to about 80% with respect to weight of the dental composition.

15. The dental composition of claim 1, the amount of the polyvinylpyrrolidone present in the dental composition is from about 1 wt. % to about 10 wt. % with respect to the weight of the dental composition.

16. The dental composition of claim 1, the dental composition exhibits the viscosity of from about 50 cP to about 1500 cP.

17. The dental composition of claim 1, the viscosity of the dental composition does not increase or increases less than about 1000 cP after the dental composition is stored at around 60° C. for about 8 weeks.

18. The dental composition of claim 1, the pH of the dental composition is substantially maintained, or decreases, or does not increase after the dental composition is stored at around 60° C. for about 4 weeks.

19. A method comprising applying dental composition of claim 1 to a tooth surface.

20. A device to apply the dental composition of claim 1 to a tooth surface, comprising:
a container configured to contain the dental composition.
